(19) United States
(12) Patent Application Publication
(10) Pub. No.: US 2022/0339623 A1
(43) Pub. Date: Oct. 27, 2022

(54) DEVICES FOR DETERMINING A TARGET NUCLEIC ACID PROFILE AND METHODS OF USE THEREOF

(71) Applicants: Frederick Edward TURNER, San Jose, CA (US); Vladimir I. SLEPNEV, San Jose, CA (US); Ronald TEDESCO, San Jose, CA (US); Curative Inc., San Dimas, CA (US)

(72) Inventors: Frederick Edward TURNER, San Dimas, CA (US); Vladimir I. SLEPNEV, Diamond Bar, CA (US); Ronald TEDESCO, San Dimas, CA (US)

(21) Appl. No.: 17/764,309

(22) PCT Filed: Sep. 29, 2020
(86) PCT No.: PCT/US2020/053297

§ 371 Date: Mar. 28, 2022

Related U.S. Application Data
(60) Provisional application No. 62/908,484, filed on Sep. 30, 2019.
(60) Provisional application No. 62/934,448, filed on Nov. 12, 2019.

(51) Int. Cl. B01L 3/00; B01L 7/00; C12Q 1/6869

(52) U.S. Cl. CPC B01L 3/5027; B01L 7/52; C12Q 1/6869; B01L 2200/10; B01L 2200/147; B01L 2300/021; B01L 2300/0681; B01L 2300/0867

(57) ABSTRACT

The present disclosure, in some aspects, provides devices useful for determining a target nucleic acid profile of a sample. The device comprises an enrichment module, a reaction module and a nucleic acid sequencer. In other aspects, the present disclosure provides kits, components and compositions (such as consumables) of the devices and methods of using the devices described herein.

### CROSS-REFERENCE TO RELATED APPLICATIONS

This application claims the priority benefit of U.S. Provisional Patent Application No. 62/908,484, filed on Sep. 30, 2019, and U.S. Provisional Patent Application No. 62/934,448, filed on Nov. 12, 2019, the disclosure of each of which is hereby incorporated herein by reference in its entirety.

### TECHNICAL FIELD

The present disclosure, in some aspects, is directed to devices for determining a target nucleic acid profile of a sample. In other aspects, the present disclosure is directed to kits, components, and compositions (such as consumables) of the devices, or for use with the devices, and methods of using the devices described herein.

### BACKGROUND

There are many different steps involved in identifying the presence of, and characterizing, a nucleic acid in sample, including enriching the nucleic acid from a sample, processing the nucleic acid, if present, for detection purposes (such as via amplification) and, optionally, sequencing. Developing compatible reagents, techniques, and devices to enrich, process, detect, and, optionally, sequence the nucleic acid from a sample on a relevant time scale (e.g., less than 4 hours) is challenging, and there currently is a lack of an integrated, all-in-one device with such capabilities on the market. Challenges with combining techniques to make feasible an integrated, sample-to-answer device include the lack of availability of an enrichment technique, and components thereof, having the sensitivity and specificity to capture low-abundance nucleic acids in real-world relevant sample volumes (e.g., from 3 mL blood), the incompatibility of some steps involved in enriching, processing, detecting, and sequencing nucleic acids (such as due to differences in the carry-over volume of one step to the next step), and, until recently, the lack of small rapid nucleic acid sequencers.

Methods and devices (including components thereof) that enable the use of high affinity nucleic acid capture probes are desirable in the field of nucleic acid enrichment. While high affinity nucleic acid capture probes are known in the art, e.g., LNA capture probes, use of high affinity nucleic acid capture probes is limited by a lack of compatible elution techniques, such as elution techniques that have the ability to elute bound target nucleic acids, are compatible with downstream analyzes, and preserve the integrity of captured target nucleic acids. For example, it has been demonstrated that capture probes comprising all LNA sequences have limited application for target nucleic acid enrichment because it was not possible to elute the captured target nucleic acids using conventional methods, such as low ionic strength solutions and heat (Nucleic Acids Research, 2004, Vol. 32, No. 7 e64). Such limitations prevent full realization of high affinity captures probes for the enrichment of target nucleic acids.

All references cited herein, including patent applications and publications, are incorporated by reference in their entirety.

### BRIEF SUMMARY

In some aspects, provided herein is a device for determining a target nucleic acid profile of a sample, the device comprising: (a) an enrichment module, wherein the enrichment module comprises an enrichment compartment; (b) a reaction module, wherein the reaction module comprises a first reaction compartment; and (c) a nucleic acid sequencer.

In some embodiments, at least two of any of (i) the enrichment module, (ii) the reaction module, and (iii) the nucleic acid sequencer are connected via a fluidic circuit.

In some embodiments, the enrichment module is connected to the reaction module via a first channel. In some embodiments, the reaction module is connected to the nucleic acid sequencer via a second channel.

In some embodiments, the enrichment compartment has a volume of about 10 mm³ to about 100 mm³. In some embodiments, the enrichment compartment contains a capture medium, wherein capture probes are associated with the capture medium, and wherein each capture probe comprises an oligonucleotide. In some embodiments, the oligonucleotide comprises one or more modified nucleotides. In some embodiments, each of the one or more modified nucleotides is selected from the group consisting of a locked nucleic acid (LNA), peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, a nucleotide with a modified sugar, base group, or backbone, or any combination thereof. In some embodiments, the capture medium is a solid support or a plurality of particles. In some embodiments, the capture probe is conjugated to a surface of the capture medium.

In some embodiments, the enrichment module is configured to cycle a fluid through the enrichment compartment.

In some embodiments, the enrichment module further comprises a first buffer channel, and wherein the first buffer channel is connected to the enrichment compartment. In some embodiments, the enrichment module further comprises a first buffer compartment, and wherein the first buffer compartment is connected to the enrichment compartment via the first buffer channel.

In some embodiments, the enrichment module further comprises an elution buffer channel, and wherein the elution buffer channel is connected to the enrichment compartment. In some embodiments, the enrichment module further comprises an elution buffer compartment, and wherein the elution buffer compartment is connected to the enrichment compartment via the elution buffer channel.

In some embodiments, the enrichment module further comprises a pH neutralization module. In some embodiments, the pH neutralization module comprises a pH sensor.

In some embodiments, the enrichment module is connected to a first waste output channel.

In some embodiments, the first reaction compartment is configured for any one or more of a reverse transcriptase step, an amplification step, an amplification product detection step, and a step for preparing nucleic acids for sequencing. In some embodiments, the reaction module further comprises a second reaction compartment, and wherein the first reaction compartment and the second reaction compartment are configured for any one or more of a reverse transcriptase step, an amplification step, an amplification product detection step, and a step for preparing nucleic acids for sequencing. In some embodiments, the reaction module further comprises a third reaction compartment, and wherein the first reaction compartment, the second reaction compartment, and the third reaction compartment are configured for any one or more of a reverse transcriptase step, an amplification step, an amplification product detection step, and a step for preparing nucleic acids for sequencing.

In some embodiments, the first reaction compartment is a reverse transcription compartment, wherein the reaction module further comprises an amplification compartment and a sequencing preparation compartment.

In some embodiments, the reverse transcription compartment is connected to the amplification compartment via a channel, and wherein the amplification compartment is connected to the sequencing preparation compartment via another channel. In some embodiments, the first reaction compartment is connected to a reagent channel.

In some embodiments, the reagent module further comprises a reagent compartment, wherein the reagent compartment is connected to the first reaction compartment via the reagent channel. In some embodiments, the first reaction compartment comprises one or more reagents useful for one or more of nucleic acid amplification, nucleic acid detection, and preparing nucleic acids for sequencing.

In some embodiments, the device further comprises a heating element, wherein the heating element is position in proximity or on the reaction module or a portion thereof.

In some embodiments, the device further comprises a temperature sensor, wherein the temperature sensor is position in proximity or on the reaction module or a portion thereof.

In some embodiments, the device further comprises a fluorescence detector.

In some embodiments, the nucleic acid sequencer is a nanopore sequencer. In some embodiments, the nanopore sequencer comprises a nanopore array that is fluidically connected to the reaction module via the second channel. In some embodiments, the nanopore sequencer comprises a sample input port.

In some embodiments, the reagent module interfaces with the sample input port of the sample input port of the nanopore sequencer.

In some embodiments, the device is configured to move a fluid from the reaction module to the nucleic acid sequencer.

In some embodiments, the device further comprises a sample preparation module. In some embodiments, the enrichment module is connected to a sample input channel. In some embodiments, the enrichment module is connected to the sample preparation module via the sample input channel.

In some embodiments, the enrichment module, the reaction module, and the nucleic acid sequencer are housed in a single cartridge. In some embodiments, the enrichment module and the reaction module are housed in a single cartridge, and the nucleic acid sequencer is housed in another cartridge.

In another aspect, provided herein is a method of determining a nucleic acid profile of a sample, the method comprising analyzing the sample using any one of the devices described herein, thereby determining the nucleic acid profile of the sample. In some embodiments, the nucleic acid profile comprises information regarding the presence, or lack thereof, of a nucleic acid, and/or information regarding the sequence of the nucleic acid.

These and other aspects and advantages of the present disclosure will become apparent from the subsequent detailed description and the appended claims. It is to be understood that one, some, or all of the properties of the various embodiments described herein may be combined to form other embodiments of the present disclosure.

### BRIEF DESCRIPTION OF THE DRAWINGS

FIG. 1 shows an exemplary workflow with representative device modules and respective exemplary capabilities.

FIG. 2 shows a schematic of select components of an exemplary device described herein.

FIG. 3 shows a schematic of an exemplary integrated sample processing-sequencing cartridge.

### DETAILED DESCRIPTION

The present disclosure is based, in part, on the inventors' unique insights for the design of an integrated, sample-to-answer device capable of enriching nucleic acids from a sample, processing the nucleic acid, if present, for detection purposes (such as via amplification), and, sequencing the amplified products. Such devices are also compatible with and take advantage of advancements in high affinity enrichment techniques, and components thereof, disclosed herein. The recent rapid progress in the development of nucleic acid sequencing components, and increases in sequencing speed, provides a unique opportunity for the development of a device that incorporates real-time sequencing with sample preparation for nucleic acid detection and characterization. Such devices have utility in, e.g., diagnostic testing and in-the-field nucleic acid analysis. The devices described herein allow for nucleic acid detection and sequencing to be performed in a rapid time frame, sufficient to enable incorporation of real-time diagnostic information into patient management decisions. For example, the devices described herein provide real-time information to guide clinical decisions, such as initiation or termination of a treatment or therapy, as well as selection of the most suitable treatment based on the acquired diagnostic information. The integrated devices described herein may also be designed on a compact scale and can be used to perform high-sensitivity tests in a laboratory or at/near the point of sample origin, such as a physician office, emergency room, or a remote or mobile location. The diagnostic information acquired from the devices described herein can then be quickly communicated to necessary personnel for use in making real-time decisions.

In some aspects, provided herein is a device for determining a target nucleic acid profile of a sample, the device comprising: (a) an enrichment module, wherein the enrichment module comprises an enrichment compartment; (b) a reaction module, wherein the reaction module comprises a first reaction compartment; and (c) a nucleic acid sequencer. In some embodiments, the device comprises a fluidic circuit comprising: (i) an enrichment module; (ii) a reaction module, wherein the reaction module is connected to the enrichment module via a first channel, and wherein the reaction module comprises a first reaction compartment; and (iii) a nucleic acid sequencer. In some embodiments, the device comprises: (a) a fluidic circuit comprising: (i) an enrichment module; and (ii) a reaction module, wherein the reaction module is connected to the enrichment module via a first channel, and wherein the reaction module comprises a first reaction compartment; and (b) a nucleic acid sequencer, wherein the nucleic acid sequencer is configured to interface with the fluidic circuit.

In other aspects, provided herein is a method of determining a target nucleic acid profile of a sample using a device described herein. In some embodiments, the method is for diagnostic testing purposes. In some embodiments, diagnostic testing is performed using a device comprising a single consumable cartridge combining reagents required to perform sample preparation, such as for liberating nucleic acids, nucleic acid amplification, and real-time sequencing. In some embodiments, the single consumable cartridge is disposable. In some embodiments, diagnostic testing is performed using a device comprising two or more consumable cartridges, wherein a single consumable cartridge performs sample preparation and amplification, which is operationally and fluidically connected to a real-time sequencing compartment allowing for the sequencing of multiple samples using the same real-time sequencer.

In some embodiments, externally prepared specimens or processes may be introduced in a single or multiple consumable cartridge(s) to bypass unwanted processing steps, saving time, reagents, costs, or enabling only specific process steps to be performed. In some embodiments, processed specimens may be exported from a single or multiple consumable cartridge(s) to bypass unwanted processing steps, saving time, reagents, costs or allowing only specific processes to be performed and to enable offline specimen processing.

Also contemplated herein are kits, components, and compositions (such as consumables) of the devices, or for use with the devices.

It will also be understood by those of ordinary skill in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### A. Definitions

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

### B. Devices for Determining a Target Nucleic Acid Profile

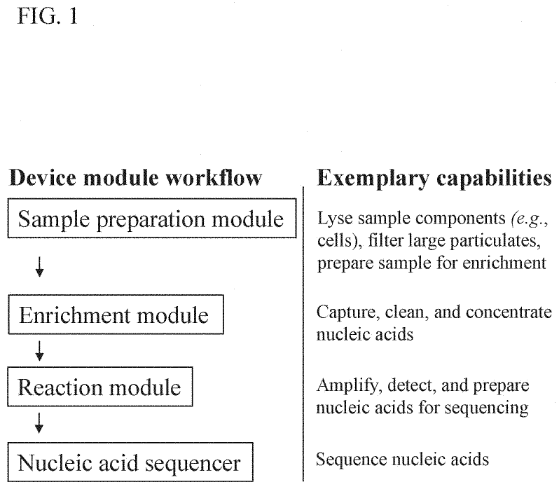

Provided herein are devices for determining a target nucleic acid profile of a sample. In some embodiments, the devices described herein are capable of integrating two or more steps of (i) enriching a nucleic acid from a sample, (ii) processing of the nucleic acid for detection purposes (and detection thereof), and (iii) sequencing of nucleic acids, including any steps for preparing nucleic acids for sequencing (such as concerning adaptor sequences and/or helper components, e.g., motor proteins). In some embodiments, the device further comprises a sample preparation module for preparing a nucleic acid in the sample for an enrichment step, e.g., performing cell lysis, enzyme inactivation, and sample filtration. As discuss in greater detail in the sections below, in some aspects, the devices described herein comprise integrated modules capable of performing nucleic acid preparation and processing steps from sample preparation to sequencing. For example, as shown in the workflow of FIG. 1, modules that the device comprises may include (i) a sample preparation module for, e.g., lysing sample components, such as cells, filtering large particulates prior to downstream workflows, and preparing the sample for nucleic acid enrichment, (ii) an enrichment module for, e.g., enriching a nucleic acid from a sample by capturing, cleaning an d concentrating the nucleic acid, (iii) a reaction module for, e.g., processing the enriched nucleic acid for detection purpose, such as PCR amplification, detection thereof, and preparation for sequencing, and (iv) a nucleic acid sequencer for, e.g., providing real-time sequencing information. In some embodiments, the device performs the steps for determining the target nucleic acid profile (such as from sample preparation to sequencing) in an automated fashion (e.g., obtaining sequence information without user intervention in the intermediate steps performed by the device). The devices described herein integrate modules for various workflows, and accordingly, in some embodiments, delimitations between different modules may not be fixed and/or may be, to an extent, arbitrarily assigned. In some embodiments, the device may perform lysis and amplification of nucleic acids prior to sequencing, wherein no target enrichment step is performed. In such an aspect, the device is performing a similar automated analysis to obtain sequence information without user intervention in the intermediate steps.

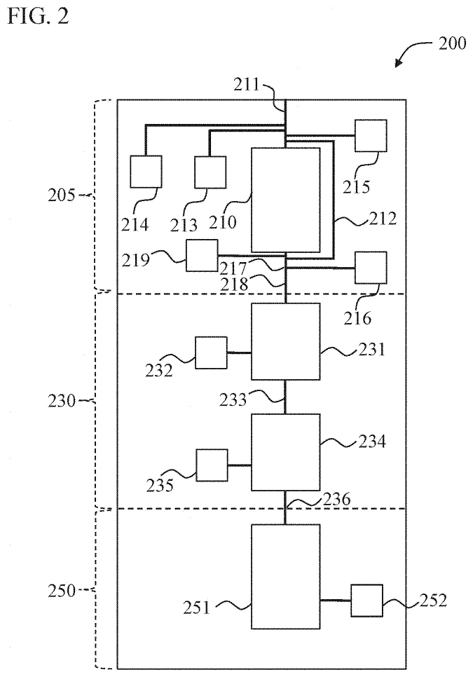

A schematic of select components of an exemplary device described herein is shown in FIG. 2. A high-level understanding of the integration of components of a device 200 can be described by illustrating modules (such as the enrichment module 205, the reaction module 230, and the nucleic acid sequencer 250). In some embodiments, the sample is introduced to the enrichment module via a sample input channel 211, and the sample is directed to an enrichment compartment 210 which contains a capture medium, wherein capture probes are associated with the capture medium, and wherein each capture probe comprises an oligonucleotide (such as the capture probes comprising a high affinity nucleotide, such as LNA, described herein). In some embodiments, the sample is cycled through the enrichment compartment 210 more than one time via a channel 212. In some embodiments, the sample, after flowing through the enrichment compartment, is directed to a waste compartment 219. In some embodiments, the device comprises wash buffer compartments 213, 214 and an elution buffer compartment 215, useful for washing and then eluting nucleic acids captured in the enrichment compartment. In some embodiments, the enrichment module 205 comprises a pH neutralization module, which may comprise a compartment 216 and a mixing junction 217, useful for neutralizing eluate from the enrichment compartment 210 (e.g., in applications requiring high pH or low pH elution steps, such as for elution with capture probes comprising a high affinity nucleotide, such as LNA, described herein). In some embodiments, the enrichment module 205 is connected to the reagent module 230 via a channel 218. In some embodiments, the regent module 230 comprises two reaction compartments 231, 234, useful for processing nucleic acids (e.g., processing the enriched nucleic acid for detection purpose, such as PCR amplification, detection thereof, and preparation for sequencing). In some embodiments, the reaction compartments 231, 234 comprise one or more reagents useful for processing nucleic acids. In some embodiments, the reaction compartments 231, 234 are connected to reagent compartments 232, 235 containing reagents via channel(s). In some embodiments, one or more reagents are contained in a channel of the fluidic circuit. In some embodiments, a first reaction compartment 231 is connected to a second reaction compartment 234 via a channel 233. In some embodiments, the reaction module 230 is connected to a nanopore compartment 251 of a nucleic acid sequencer 250 via a channel 236. In some embodiments, the nucleic acid sequencer 250 further comprises a priming compartment 252 containing a priming reagent.

The devices described herein are configured to move fluid in a module and between integrated modules in a controlled manner. For example, in some embodiments, the device is configured to move a fluid from one compartment to another compartment. In some embodiments, the compartment is a region of a portion of the device. In some embodiments, the compartment is not fully enclosed, and, e.g., such compartments may encompass a surface or a well. It is appreciated that there are numerous methods and device features for moving a fluid. Such methods and device features encompass, for example, channel-based fluidics (e.g., use of pneumatic forces to move fluid in a microfluidic device), chemical, electrical, wave-based, and temperature-based fluidics, and mechanical dispensing fluidics. The devices described herein may encompass one or more methods and device features for moving a fluid, including combinations thereof. For example, in some embodiments, the devices described herein comprise a fluidic circuit comprising the integrated modules and a nucleic acid sequencer, wherein the device is configured to move a fluid through the fluidic circuit in a controlled manner. In some embodiments, the device further comprises one or more features for sample preparation and sample handling, wherein the additional features for sample preparation and sample handling are connected to the fluidic circuit, and wherein the device is configured to move a fluid through the fluidic circuit in a controlled manner. In some embodiments, the fluidic circuit is a microfluidic circuit.

In some aspects, the devices described herein incorporate temperature control elements maintaining different compartments at the same and/or different temperatures over time. For example, in some embodiments, sample enrichment and amplification methodologies may be performed at elevated temperatures (i.e., above ambient temperatures), whereas the nucleic acid sequencer is maintained at a temperature close to ambient temperatures. Various means are understood, and encompassed by the description, to provide optimal temperature to different parts of the device/cartridge(s) and to isolate specific temperature ranges to the different areas of the device/cartridge(s).

In some aspects, the devices described herein are configured to be compact, such as for use as a handheld, bench top, or point-of-care device. Additionally, the devices described herein encompass a number of cartridge-based configurations. For example, in some embodiments, the fluidic circuit comprising the integrated modules for enrichment and processing, and the nucleic acid sequencer are configured as a single cartridge (such as a disposable cartridge). In some embodiments, the fluidic circuit comprising the integrated modules for enrichment and processing is on a first cartridge and the nucleic acid sequencer is on a second cartridge. In some embodiments, the device comprises two or more cartridges comprising a fluidic circuit comprising the integrated modules for enrichment and processing.

In some embodiments, the device, or method of use thereof, and/or selection of components thereof, are customized to the specifications of a sample or the needs of a desired nucleic acid profile from the sample. In some embodiments, the term profile, such as used in reference to a nucleic acid profile, refers to one or more information/data points associated with one or more nucleic acids in a sample. In some embodiments, the profile may include different types of information, e.g., index scores, expression levels, presence versus absence, identification information, and attribute information. In some embodiments, the nucleic acid profile comprises information regarding the presence (or lack thereof) of a nucleic acid in a sample. In some embodiments, the nucleic acid profile comprises quantitative information regarding the presence of a nucleic acid in a sample. In some embodiments, the nucleic acid profile comprises information regarding the sequence of a nucleic acid in a sample. In some embodiments, the nucleic acid profile comprises information regarding the presence (including quantitative measurements thereof) of a nucleic acid in a sample and the sequence of a nucleic acid in a sample.

From the disclosure of the present application, it will be readily appreciated that the devices described herein may encompass numerous and different configurations of a selection of components and features disclosed herein. In some embodiments, the device comprises a set of features, such as a subset of all features disclosed herein, suitable to obtain a target nucleic acid profile of a specific sample, and/or to obtain desired information from the sample. Discussion of features of the devices described herein in a modular fashion is not to be construed as limiting the combinations of features and capabilities encompassed by the devices of the present disclosure. In some embodiments, a single feature is configured for more than one purpose.

i. Enrichment Modules

The enrichment modules described herein are configured for enriching a nucleic acid from a sample, if present therein. Accordingly, in some embodiments, the enrichment modules are configured to capture a nucleic acid from a sample (such samples may have variable volumes and include sample sizes of, e.g., 1-10 mL), wash the captured nucleic acid with one or more wash buffers (e.g., using wash buffer volumes of about 1-5 mL), and elute the nucleic acid in a suitable volume for further processing (such eluate volumes may be, e.g., 5-500 μL). In some embodiments, the ability to interrogate a large volume of sample (e.g., 1-10 mL) using a capture device of a minimal volume (e.g., about 0.01-0.1 mL) is of particularly importance for significant enrichment of target nucleic acids before amplification and detection steps. The devices description in the present disclosure encompass integration of components to manage the differences in fluid volumes.

In some embodiments, the enrichment module comprises an enrichment compartment. In some embodiments, the enrichment compartment has a volume of about 10 mm³ to about 500 mm³, such as about 10 mm³ to about 200 mm³, about 20 mm³ to about 100 mm³, about 50 mm³ to about 200 mm³, about 50 mm³ to about 150 mm³, or about 250 mm³ to about 500 mm³. In some embodiments, the enrichment compartment has a volume of at least about 10 mm³, such as at least about any of 20 mm³, 30 mm³, 40 mm³, 50 mm³, 60 mm³, 70 mm³, 80 mm³, 90 mm³, 100 mm³, 125 mm³, 150 mm³, 175 mm³, 200 mm³, 250 mm³, 300 mm³, 350 mm³, 400 mm³, 450 mm³, or 500 mm³. In some embodiments, the enrichment compartment has a volume of less than about 500 mm³, such as less than about any of 450 mm³, 400 mm³, 350 mm³, 300 mm³, 250 mm³, 200 mm³, 175 mm³, 150 mm³, 125 mm³, 100 mm³, 90 mm³, 80 mm³, 70 mm³, 60 mm³, 50 mm³, 40 mm³, 30 mm³, 20 mm³, or 10 mm³. In some embodiments, the enrichment compartment has a volume about any of 10 mm³, 20 mm³, 30 mm³, 40 mm³, 50 mm³, 60 mm³, 70 mm³, 80 mm³, 90 mm³, 100 mm³, 125 mm³, 150 mm³, 175 mm³, 200 mm³, 250 mm³, 300 mm³, 350 mm³, 400 mm³, 450 mm³, or 500 mm³.

The enrichment compartments of the devices described herein may be a number of different shapes, and have any number of features configured for efficient fluid flow therethrough, nucleic acid capture, washing, and elution. In some embodiments, the enrichment compartment, or a portion thereof, is a column. In some embodiments, the enrichment compartment, or a portion thereof, is a cuboid. In some embodiments, the enrichment compartment, or a portion thereof, is rectangular.

In some embodiments, the enrichment compartment is configured for a sample flow rate of about 0.05 mL/minute to about 1 mL/minute, such as about 0.05 mL/minute to about 0.3 mL/minute, about 0.1 mL/minute to about 0.3 mL/minute, or about 0.2 mL/minute to about 0.8 mL/minute. In some embodiments, the enrichment compartment is configured for a sample flow rate of at least about 0.001 mL/minute, such as at least about any of 0.05 mL/minute, 0.1 mL/minute, 0.15 mL/minute, 0.2 mL/minute, 0.25 mL/minute, 0.3 mL/minute, 0.35 mL/minute, 0.4 mL/minute, 0.5 mL/minute, 0.6 mL/minute, 0.7 mL/minute, 0.8 mL/minute, 0.9 mL/minute, or 1 mL/minute. In some embodiments, the enrichment compartment is configured for a sample flow rate of less than about 1 mL/minute, such as a less than about any of 0.9 mL/minute, 0.8 mL/minute, 0.7 mL/minute, 0.6 mL/minute, 0.5 mL/minute, 0.4 mL/minute, 0.35 mL/minute, 0.3 mL/minute, 0.25 mL/minute, 0.2 mL/minute, 0.15 mL/minute, 0.1 mL/minute, or 0.05 mL/minute. In some embodiments, the enrichment compartment is configured for a sample flow rate of about any of 0.05 mL/minute, 0.1, mL/minute, 0.15 mL/minute, 0.2 mL/minute, 0.25 mL/minute, 0.3 mL/minute, 0.35 mL/minute, 0.4 mL/minute, 0.5 mL/minute, 0.6 mL/minute, 0.7 mL/minute, 0.8 mL/minute, 0.9 mL/minute, or 1 mL/minute.

In some embodiments, the enrichment module is configured for single direction flow through the enrichment compartment. In some embodiments, the enrichment module is configured for bidirectional flow through the enrichment compartment. In some instances, for descriptive purposes, it is helpful to assign a directionality to features of a module, e.g., an enrichment compartment, and such directionality may be based on the direction of elution through the enrichment compartment. As described herein, the downstream portion of an enrichment compartment is the portion wherein the eluate (which in some instances may comprise a captured and eluted nucleic acid) leaves the enrichment compartment.

In some embodiments, the enrichment module is configured to pass the sample through the enrichment compartment more than one time. In some embodiments, wherein the enrichment module is configured for single direction flow through the enrichment compartment, the enrichment module comprises a cycling channel with an opening located in the downstream portion of the enrichment compartment to direct the sample (after it passes through the enrichment compartment) back to the upstream portion of the enrichment compartment. In some embodiments, the cycling channel comprises (or is connected to) a compartment for holding the sample (or a portion thereof) during the sample cycling process. In some embodiments, wherein the enrichment module is configured for bidirectional flow through the enrichment compartment, the enrichment module comprises a compartment connected to the downstream portion of the enrichment compartment to hold the sample (after it passes through the enrichment compartment). In some embodiments, wherein the enrichment module is configured for bidirectional flow through the enrichment compartment, the device is configured to direct the sample back through the enrichment compartment.

In some embodiments, the device is configured to subject the contents in an enrichment module or a portion thereof, such as an enrichment compartment, to a temperature of about 20° C. to about 100° C. In some embodiments, the device is configured to subject the contents in an enrichment module or a portion thereof, such as the enrichment compartment, to a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., 95° C., or 100° C.

In some embodiments, the enrichment compartment contains a capture medium, wherein capture probes are associated with the capture medium, and wherein each capture probe comprises an oligonucleotide. In some embodiments, the capture medium is a solid support or a solid surface. In some embodiments, the capture medium is a matrix. In some embodiments, the capture medium is resistant to shrinking and/or swelling, such as during exposure to buffers of various ion strength and/or pH. In some embodiments, the capture medium is a plurality of particles, such as beads. In some embodiments, the capture medium comprises a plurality of magnetic beads, such as polymer-coated magnetic beads. In some embodiments, the largest dimension of each of the particles is about 5 μm to about 300 μm, such as about 25 μm to about 100 μm, about 50 μm to about 100 μm, about 50 μm to about 90 μm, about 50 μm to about 250 μm, or about 100 μm to about 250 μm. In some embodiments, the largest dimension of each of the particles is at least about 5 μm, such as at least about any of 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 225 μm, 250 μm, 275 μm, or 300 μm. In some embodiments, the largest dimension of each of the particles is less than about 300, such as less than about any of 275 μm, 250 μm, 225 μm, 200 μm, 190 μm, 180 μm, 170 μm, 160 μm, 150 μm, 140 μm, 130 μm, 120 μm, 110 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, or 5 μm. In some embodiments, the largest dimension of each of the particles is about any of 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 225 μm, 250 μm, 275 μm, or 300 μm.

In some embodiments, the capture medium is composed of a material comprising a polyacrylic or polymethacrylic polymer. In some embodiments, the capture medium is composed of a material comprising a polystyrene or crosslinked polystyrene.

In some embodiments, the capture medium is a 5′-dimethoxytrityl-adenosine-2′,3′-diacetate-N-linked-polymeric support. In some embodiments, the capture medium is an Oligo-Affinity Support (PS) (Glen Research, Sterling, Va.).

In some embodiments, the enrichment module further comprises one or more features configured to contain the capture medium in the enrichment module or a portion thereof, such as an enrichment compartment. In some embodiments, the feature configured to contain the capture medium in the enrichment module is a filter, frit, or a structure (e.g., a constriction smaller than the size of the capture medium) of the enrichment module or a portion thereof, such as an enrichment compartment, or a mixture thereof. In some embodiments, the feature configured to contain the capture medium in the enrichment module, or a portion thereof, is positioned at the downstream portion of the enrichment compartment. In some embodiments, the feature configured to contain the capture medium in the enrichment module, or a portion thereof, is positioned at the downstream and upstream portion of the enrichment compartment. In some embodiment, the feature configured to contain the capture medium in the enrichment module, or a portion thereof, is position at any opening the feature, such as at any channel interface with the feature.

In some embodiments, the capture probe comprises a hybridizing portion, wherein the hybridizing portion of the capture probes comprises a nucleic acid sequence, such as two or more consecutive nucleotides, capable of hybridizing to a target nucleic acid or a portion thereof. In some embodiments, the hybridizing portion of a capture probe comprises about 5 nucleotides to about 50 nucleotides, such as about 5 nucleotides to about 25 nucleotides, about 10 nucleotides to about 20 nucleotides, or about 20 nucleotides to about 40 nucleotides. In some embodiments, the hybridizing portion of a capture probe comprises at least about 5 nucleotides, such as at least about any of 10 nucleotides, 15 nucleotides, 20 nucleotides, 25 nucleotides, 30 nucleotides, 35 nucleotides, 40 nucleotides, 45 nucleotides, or 45 nucleotides. In some embodiments, the hybridizing portion of a capture probe comprises less than about 45 nucleotides, such as less than about any of 40 nucleotides, 35 nucleotides, 30 nucleotides, 25 nucleotides, 20 nucleotides, 15 nucleotides, or 10 nucleotides. In some embodiments, the hybridizing portion of a capture probe comprises any of 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides.

In some embodiments, the hybridizing portion of a capture probe comprises one or more modified nucleotides. In some embodiments, the modified nucleotide is an analog that obeys Watson-Crick base pairing. In some embodiments, the modified nucleotide is a derivative that obeys Watson-Crick base pairing. In some embodiments, the modified nucleotide hybridizes to a complementary nucleotide, such as a naturally occurring nucleotide, with stronger affinity than a naturally occurring complementary nucleotide. In some embodiments, the modified nucleotide is selected from the group consisting of a locked nucleic acid (LNA), a peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, and a nucleotide with one or more sugar, base group, or backbone modifications.

In some embodiments, the nucleotides of a hybridizing portion of a capture probe comprises about 40% to about 100% modified nucleotides, such as about 40% to about 80% modified nucleotides, about 60% to about 90% modified nucleotides, about 75% to about 100% modified nucleotides, or about 90% to about 100% modified nucleotides. In some embodiments, the nucleotides of a hybridizing portion of a capture probe comprises at least about 40%, such as at least about any of 45%, 50%, 55%, 60%, 65%, 70%, 75%, 80%, 85%, 90%, 95%, 96%, 97%, 98%, 99%, or 100%, modified nucleotides. In some embodiments, the nucleotides of a hybridizing portion of a capture probe comprise about any of 80% modified nucleotides, 81% modified nucleotides, 82% modified nucleotides, 83% modified nucleotides, 84% modified nucleotides, 85% modified nucleotides, 86% modified nucleotides, 87% modified nucleotides, 88% modified nucleotides, 89% modified nucleotides, 90% modified nucleotides, 91% modified nucleotides, 92% modified nucleotides, 93% modified nucleotides, 94% modified nucleotides, 95% modified nucleotides, 96% modified nucleotides, 97% modified nucleotides, 98% modified nucleotides, 99% modified nucleotides, or 100% modified nucleotides.

In some embodiments, the hybridizing portion of a capture probe is designed to have a desired affinity for a target nucleic acid or a portion thereof. The affinity of the a hybridizing portion for a target nucleic acid or a portion thereof may be tuned based on, e.g., length (e.g., number of nucleotides), number of consecutive nucleotides, degree of complementarity to a target sequence, inclusion of modified nucleotides, inclusion of a space and/or a linker, such as spacer or linker bases, inclusion of mismatched nucleotides, and amount of modified high affinity nucleotides. In some embodiments, the hybridizing portion of a capture probe is designed to have a desired affinity for a target nucleic acid, or a portion thereof, in a specific condition (such as in a denaturing condition, e.g., a solution of 4M guanidinium salts, and/or an elevated temperature, e.g., about 30° C. to about 60° C.). In some embodiments, the capture probe comprises more than one hybridizing portion, such as any of 2, 3, 4, or 5 hybridizing portions.

In some embodiments, the hybridizing portion of a capture probe is complementary to a target nucleic acid or a portion thereof. In some embodiments, the hybridizing portion of a capture probe has at least about 60%, such as at least about any of 65%, 70%, 75%, 80%, 85%, 90%, 95%, 96%, 97%, 98%, or 99%, sequence complementarity to a target nucleic acid or a portion thereof.

In some embodiments, the desired affinity for a target nucleic acid or a portion thereof is based on a melting temperature threshold affinity. The threshold affinity for oligonucleotide hybridization may be estimated using T. (melting temperature) calculation. It is noted that T. calculation is highly dependent on, e.g., ionic conditions, on the nature of cations, presence of denaturants, and the extent of incorporation of high affinity bases (e.g., LNA) into the capture probe. Thus, a capture probe designed to have a desired affinity for a target nucleic acid or a portion thereof will have a desired affinity for a target nucleic acid or a portion thereof in a relevant condition. In some embodiments, empirically, the capture probe binding to the target nucleic acid under conditions denaturing conditions (e.g., 4 M guanidinium salt) can be measured and used as a threshold affinity.

In some embodiments, the capture probe comprising one or more nucleotide analogs is a polynucleotide or oligonucleotide containing one or more LNA monomers and a variable number of naturally occurring nucleotides or their analogues, such as 7-deazaguanosine or inosine, and is sufficiently complementary to hybridize with the target nucleic acid such that stable and specific binding occurs to form a complex between the target and the complementary nucleic acid under the hybridization conditions. In some embodiments, the capture probe sequence need not reflect the exact sequence of the target nucleic acid. For example, a non-complementary nucleotide fragment may be attached to a complementary nucleotide fragment or alternatively, non-complementary bases or longer sequences can be interspersed into the complementary nucleic acid, provided that the complementary nucleic acid sequence has sufficient complementarity with the sequence of the target nucleic acid to hybridize therewith, forming a hybridization complex and further is capable of immobilizing the target nucleic acid to a solid support as will be described in further detail below.

In some embodiments, the capture probe comprises a linker or a spacer moiety. In some embodiments, the capture probe is attached to a partner of a binding group (e.g., biotin/avidin, fluorescein or carboxyfluorescein/albumin, magnetic micro-particle, a component of a click chemistry group), such as via the linker or the spacer moiety. In some embodiments, the capture probe is associated with a capture medium, e.g., a solid phase or particle. In some embodiments, the capture probe is bound, such as covalently bound, to a capture medium, e.g., a solid phase or particle. In some embodiments, the capture probe is associated with or bound to a capture medium via the binding group.

In some embodiments, the capture probe comprises a linker. In some embodiments, the linker comprises a nucleotide, such as a nucleotide that will not hybridize to a target nucleic acid or a portion thereof when the hybridizing portion of the capture probe is hybridized thereto. In some embodiments, the linker comprises a polymer, such as a linear polymer. In some embodiments, the polymer is based on a nucleic acid backbone structure. In some embodiments, the linker comprises phosphoramidite, such as phosphoramidite C3, phosphoramidite 9, phosphoramidite C12, or phosphoramidite 18. In some embodiments, the length of the linker is equal to the length of a linear single-stranded nucleic acid that is about 1 nucleotide to about 100 nucleotides, such as about 1 nucleotide to about 50 nucleotides, about 5 nucleotides to about 50 nucleotides, or about 5 nucleotides to about 100 nucleotides.

In some embodiments, the capture probe comprises a spacer. In some embodiments, the spacer separates, such as sits between on a linear nucleic acid, two parts of a hybridizing portion of a capture probe. In some embodiments, the spacer separates a hybridizing portion and a linker of a capture probe. In some embodiments, the spacer comprises non-hybridizing nucleotides, e.g., nucleotides selects based on the target nucleic acid sequence. In some embodiments, the length of the spacer is equal to the length of a linear single-stranded nucleic acid that is about 1 nucleotide to about 50 nucleotides, such as about 5 nucleotides to about 50 nucleotides.

In some embodiments, the affinity of a capture probe can be tuned, such as further tuned, by incorporating one or more spacers and/or linkers that do not hybridize to a target nucleic acid, wherein the one or more spacers and/or linkers are positioned in the capture probe to separate hybridizing portions of the capture probe. In some embodiments, for a capture probe comprising more than one hybridizing portions separated by a linker and/or a spacer, the segmented hybridizing portions contain complementary sequences to the target nucleic acid, the hybridizing portions comprising one or more LNA and/or DNA nucleotides. In some embodiments, for a capture probe comprising more than one hybridizing portions separated by a linker and/or a spacer, the hybridizing portions comprise a sequence of about 6 to about 20 nucleotides (such as LNA), and wherein the hybridizing portions are separated by a spacer and/or a linker with a length that is equal to the length of a linear single-stranded nucleic acid that is about 5 nucleotides to about 20 nucleotides. In some embodiments, the capture probe comprises two hybridizing portions separated by a linker and/or a spacer, wherein each of the hybridizing portions comprise a sequence of about 6 to about 20 nucleotides (such as LNA), and wherein the hybridizing portions are separated by a spacer and/or a linker with a length that is equal to the length of a linear single-stranded nucleic acid that is about 5 nucleotides to about 20 nucleotides.

In some embodiments, the capture probe is associated with a surface of a capture medium. In some embodiments, the capture probe is attached to a surface of a capture medium via a linker, wherein the length of the linker is equal to the length of a linear single-stranded nucleic acid that is about 5 nucleotides to about 100 nucleotides. In some embodiments, the capture probe is attached (such as conjugated) to a surface of a capture medium. In some embodiments, the capture probe comprises one or more branching nucleotides, wherein multiple target binding sequences are linked to the single attachment site at the surface of the capture medium.

In some embodiments, the density of the capture probes is about 5 nanomole of capture probe per mL of capture medium to about 250 nanomole of capture probe per mL of capture medium, such as about 10 nanomole of capture probe per mL of capture medium to about 100 nanomole of capture probe per mL of capture medium. In some embodiments, the density of the capture probes is at least about 10 nanomole per mL of capture medium, such as at least about any of 20, such as at least about any of 30, 40, 50, 60, 70, 80, 90, 100, 125, 150, 175, 200, 225, or 250, nanomole per mL of capture medium.

In some embodiments, the capture medium in an enrichment module has a single species of capture probes (e.g., capture probes comprising identical hybridizing portions) associated therewith. In some embodiments, the capture medium in an enrichment module has two or more species of capture probes (e.g., capture probes capable of hybridizing different target nucleic acids) associated therewith. The configuration of a capture medium (including the capture probe(s) associated with the capture medium) may be guided by the use of the device and/or the desired target nucleic acid profile to be obtained from the sample.

In some embodiments, the enrichment module comprises two or more enrichment compartments. In some embodiments, wherein an enrichment module comprises two or more enrichment compartments, each enrichment compartment comprises a capture medium having capture probes with affinity for different target nucleic acids associated therewith.

In some embodiments, the enrichment module is configured so that the capture medium may be washed with one or more buffers after a sample is passed therethrough. Accordingly, in some embodiments, the enrichment module further comprises a first buffer channel, wherein the first buffer channel is connected to the enrichment compartment (such as at an upstream portion of the enrichment compartment). In some embodiments, the enrichment module further comprises a first wash buffer compartment, wherein the first wash buffer compartment is connected to the enrichment compartment via a first buffer channel. In some embodiments, the device comprises an enrichment module configured to connect to a first wash buffer compartment, wherein the first wash buffer compartment is connected to the enrichment module via a first wash buffer channel (e.g., as configured for a device with a wash buffer cartridge that is inserted into the device). In some embodiments, the first buffer channel is configured to direct a wash buffer to the upstream portion of the enrichment compartment. In some embodiments, the devices described herein, and methods of use thereof, are configured for use of a plurality of consecutive wash buffers, such as 2, 3, or 4 or more. In some embodiments, the first wash buffer represents a buffer, similar in composition to the sample-containing buffer. In some embodiments, additional wash buffer compositions can be used to reduce residual contamination by components of the sample and to improve chemical compatibility with the elution buffer (e.g., prevent precipitation of buffer components). Volume of wash buffers may be proportional to the size of the enrichment compartment and represent, e.g., 1-200 times the volume of the enrichment compartment or capture medium. In some embodiments, wherein the capture medium is forming a microcolumn contained between two filters within the volume of the enrichment compartment, the volume of each wash buffer is within about 5 to about 100 times the volume of the capture medium. In some embodiments, wherein the capture medium comprises a plurality of magnetic beads or particles, the volume of each wash buffer is within about 1 to about 10 times the volume of the enrichment compartment. In some embodiments, the components of the device configured to direct a wash buffer to the enrichment compartment are configured based on the volume of wash buffer selected for use therewith.

In some embodiments, the enrichment module comprises features configured to direct a plurality of wash buffers to an enrichment compartment. In some embodiments, the enrichment module comprises a single buffer channel connected to an enrichment compartment. In some embodiments, the single buffer channel is connected to more than one wash buffer compartments. In some embodiments, the enrichment module comprises more than one buffer channel connected to an enrichment compartment. In some embodiments, the enrichment module comprises more than one buffer compartments, wherein each buffer compartment is connected to an enrichment compartment via a buffer channel. In some embodiments, the plurality of buffer compartments are connect to an enrichment compartment via one or more buffer channels.

In some embodiments, the enrichment compartment is configured for a wash buffer flow rate of about 0.05 mL/minute to about 1 mL/minute, such as about 0.05 mL/minute to about 0.3 mL/minute, about 0.1 mL/minute to about 0.3 mL/minute, or about 0.2 mL/minute to about 0.8 mL/minute. In some embodiments, the enrichment compartment is configured for a wash buffer flow rate of at least about 0.001 mL/minute, such as at least about any of 0.05 mL/minute, 0.1 mL/minute, 0.15 mL/minute, 0.2 mL/minute, 0.25 mL/minute, 0.3 mL/minute, 0.35 mL/minute, 0.4 mL/minute, 0.5 mL/minute, 0.6 mL/minute, 0.7 mL/minute, 0.8 mL/minute, 0.9 mL/minute, or 1 mL/minute. In some embodiments, the enrichment compartment is configured for a wash buffer flow rate of less than about 1 mL/minute, such as a less than about any of 0.9 mL/minute, 0.8 mL/minute, 0.7 mL/minute, 0.6 mL/minute, 0.5 mL/minute, 0.4 mL/minute, 0.35 mL/minute, 0.3 mL/minute, 0.25 mL/minute, 0.2 mL/minute, 0.15 mL/minute, 0.1 mL/minute, or 0.05 mL/minute. In some embodiments, the enrichment compartment is configured for a wash buffer flow rate of about any of 0.05 mL/minute, 0.1, mL/minute, 0.15 mL/minute, 0.2 mL/minute, 0.25 mL/minute, 0.3 mL/minute, 0.35 mL/minute, 0.4 mL/minute, 0.5 mL/minute, 0.6 mL/minute, 0.7 mL/minute, 0.8 mL/minute, 0.9 mL/minute, or 1 mL/minute.

In some embodiments, the enrichment module is configured so that the capture medium may be subjected to elution after sample is passed therethrough. Accordingly, in some embodiments, the enrichment module further comprises an elution buffer channel, wherein the elution buffer channel is connected to the enrichment compartment. In some embodiments, the enrichment module further comprises an elution buffer compartment, wherein the elution buffer compartment is connected to the enrichment compartment via an elution buffer channel. In some embodiments, the device comprises an enrichment module configured to connect an elution buffer compartment, wherein the elution buffer compartment is connected to the enrichment module via an elution buffer channel (e.g., as configured for a device with an elution buffer cartridge that is inserted into the device). In some embodiments, the elution buffer channel is configured to direct an elution buffer to the upstream portion of the enrichment compartment. In some embodiments, the volume of the elution buffer is within about 1 to about 50 times the volume of the capture medium or enrichment compartment. In some embodiments, the components of the device configured to direct an elution buffer to the enrichment compartment are configured based on the volume of elution buffer selected for use therewith. In some embodiments, the elution step, and component associated therewith, is omitted and nucleic acid amplification takes places in the presence of the capture medium.

In some embodiments, the enrichment module further comprises a pH neutralization module. In some embodiments, the pH neutralization module comprises a pH buffer channel, wherein the pH buffer channel interfaces with a portion of the device downstream of the enrichment compartment. In some embodiments, the pH neutralization module comprises a pH buffer channel connected to the first channel connecting an enrichment module and a reaction module. In some embodiments, the pH neutralization module comprises a pH neutralization compartment, wherein the pH neutralization compartment is positioned downstream of the enrichment compartment. In some embodiments, the pH neutralization compartment comprises a reagent for neutralizing the pH of an elution buffer. In some embodiments, the reagent for neutralizing the pH of an elution buffer is a liquid or solid (such as a dried reagent). In some embodiments, the pH neutralization module comprises a mixing feature, such as a mixing circuit or mixing junction. In some embodiments, the pH neutralization module comprises a pH sensor. An example of a pH sensor is disclosed in Yamada et al., Sensors, 17, 2017, which is hereby incorporated by reference in its entirety.

In some embodiments, the enrichment module is connected to a first waste output channel. In some embodiments, the enrichment module is connected to a waste compartment, wherein the waste compartment is connected to the enrichment module, such as downstream of the enrichment compartment, via a first waste output channel. In some embodiments, the enrichment module is connected to a plurality of waste compartments. In some embodiments, the waste compartment contains all the sample and wash buffers passing through the enrichment compartment. In some embodiments, the volume of a waste compartment may therefore accommodate about 1 to about 1000 times the volume of the enrichment compartment or capture medium. In some embodiments a waste compartment is located outside of cartridge element and waste collection is performed for a plurality of cartridge elements.

In some embodiments, the enrichment compartment contains capture medium in the form of magnetic beads or particles. In some embodiments, the device is configured with a magnetizing device such that the magnetic capture medium is held in a defined space or can be moved, e.g., in and out of the enrichment compartment. In some embodiments, the enrichment compartment is configured to receive a sample, or a portion thereof, comprising the capture medium in the form of magnetic beads or particles. In some embodiments, the enrichment compartment is configured to form a concentration of a capture medium in the form of magnetic beads or particles.

In some embodiments, the device comprises a heating element, wherein the heating element is configured to be in proximity to an enrichment module or a portion thereof (such as an enrichment compartment. In some embodiments, the enrichment module comprises a heating element, wherein the heating element is configured to be in proximity to a feature thereof. In some embodiments, the heating element is a transistor-based heating element. In some embodiments, the heating element is a resistor-based heating element.

ii. Sample Preparation Modules

In some aspects, the devices provided herein further comprise one or more features for sample preparation and sample handling, e.g., sample introduction, cell lysis, enzyme inactivation, and sample filtration. In some embodiments, the sample preparation module comprises a sample input port. In some embodiments, the sample preparation module comprises one or more compartments for preparing the sample for nucleic acid enrichment. In some embodiments, the enrichment module is connected to a sample input channel. In some embodiments, the enrichment module is connected to a sample input channel, wherein the sample input channel directs the sample to the upstream end of an enrichment compartment.

In some embodiments, the device is configured to subject a sample in the sample preparation module to a condition useful for lysing components of the sample, such as a cells. In some embodiments, the device comprises a feature for exposing a sample in the sample preparation module to one or more of sonication, surface acoustic waves (SAW), freeze/thaw cycling, and heating. In some embodiments, the device is configured to subject a sample in the sample preparation module to a temperature of about 40° C. to about 100° C. In some embodiments, the device is configured to subject a sample in the sample preparation module to a temperature of at least about 40° C., such as at least about any of 45° C., 50° C., 55° C., 60° C., 65° C., 70° C., 75° C., 80° C., 85° C., 90° C., 95° C., or 100° C.

In some embodiments, the sample preparation module comprises one or more sample filters. In some embodiments, the sample filter is a filter having a pore size of about 1 μm to about 50 μm. In some embodiments, the sample filter is a filter having a pore size of less than about 50 μm, such as less than about any of 45 μm, 40 μm, 35 μm, 30 μm, 25 μm, 20 μm, 15 μm, 10 μm, or 5 μm. In some embodiments, the sample filter is a filter having a pore size of about any of 1 μm, 2 μm, 3 μm, 4 μm, 5 μm, 10 μm, 15 μm, 20 μm, 21 μm, 22 μm, 23 μm, 24 μm, 25 μm, 30 μm, 35 μm, 40 μm, 45 μm, or 50 μm. In some embodiments, the sample filter has a thickness of about 0.1 mm to about 5 mm. In some embodiments, the sample filter has a thickness of at least about 0.1 mm, such as at least about 0.2 mm, 0.3 mm, 0.4 mm, 0.5 mm, 0.6 mm, 0.7 mm, 0.8 mm, 0.9 mm, 1 mm, 1.25 mm, 1.5 mm, 1.75 mm, 2 mm, 2.5 mm, 3 mm, 3.5 mm, 4 mm, 4.5 mm, or 5 mm. In some embodiments, the sample filter has a thickness of less than about 5 mm, such as less than about any of 4.5 mm, 4 mm, 3.5 mm, 3 mm, 2.5 mm, 2 mm, 1.75 mm, 1.5 mm, 1.25 mm, 1 mm, 0.9 mm, 0.8 mm, 0.7 mm, 0.6 mm, 0.5 mm, 0.4 mm, 0.3 mm, 0.2 mm, or 0.1 mm.

iii. Reaction Modules

The reaction modules described herein are configured for processing a nucleic acid enriched from a sample, if present therein, for purposes of detection (such as via amplification) and subsequent sequencing.

In some embodiments, the reaction module is configured to subject a nucleic acid to the steps of: (i) reverse transcription, (ii) amplification and product detection, and (iii) preparation for nucleic acid sequencing. In some embodiments, the reaction module of a device described herein is configured with individual compartments to perform each step involved in nucleic acid processing. For example, in some embodiments, the reaction module comprises a reverse transcription compartment, an amplification compartment (e.g., such as a compartment suitable for detection of amplified nucleic acids), and a sequencing preparation compartment. In some embodiments, the reaction module of a device described herein is configured with a compartment suitable for subjecting a nucleic acid to two or more steps of: (i) reverse transcription, (ii) amplification and product detection, and (iii) preparation for nucleic acid sequencing. For example, in some embodiments, the reaction module comprises a compartment configured for reverse transcription, amplification, and detection, and another compartment configured for preparation of nucleic acids for nucleic acid sequencing. In some embodiments, the reaction module comprises a compartment configured for reverse transcription, and another compartment for amplification, detection, and preparation of nucleic acids for nucleic acid sequencing. In some embodiments, the reaction module comprises a compartment configured for reverse transcription coupled with amplification and detection, and another compartment for preparation of nucleic acids for nucleic acid sequencing. In some embodiments, the reaction module of a device described herein is configured with a compartment suitable for subjecting a nucleic acid to the steps of: (i) reverse transcription, (ii) amplification and product detection, and (iii) preparation for nucleic acid sequencing. For example, in some embodiments, the reaction module comprises a first compartment configured for reverse transcription, amplification, detection, and preparation of nucleic acids for nucleic acid sequencing.

In some embodiments, the volume of a compartment of a reaction module is about 5 μL to about 100 μL. In some embodiments, the volume of a compartment of a reaction module is at least about 1 μL, such as at least about any of 2 μL, 3 μL, 4 μL, 5 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, or 100 μL. In some embodiments, the volume of a compartment of a reaction module is less than about 100 μL, such as less than about any of 95 μL, 90 μL, 85 μL, 80 μL, 75 μL, 70 μL, 65 μL, 60 μL, 55 μL, 50 μL, 45 μL, 40 μL, 35 μL, 30 μL, 25 μL, 20 μL, 15 μL, 10 μL, 5 μL, 4 μL, 3 μL, 2 μL, or 1 μL. In some embodiments, the volume of a compartment of a reaction module is about 0.5 to about 5 times the volume of the enrichment compartment or capture medium.

The nucleic acid processing steps that the reaction modules described herein are configured to perform require various regents and reaction conditions. The devices described herein are configured to supply and/or control access to the necessary regents and reaction conditions. Various configurations and combinations of features of the reaction modules are possible and within the scope of the devices described herein. In some embodiments, the reagent is a liquid or solid (such as a dried reagent). In some embodiments, the reaction module is configured to hold the necessary reagents in a compartment in which the processing occurs, e.g., reverse transcription reagents held in a reverse transcription compartment or a channel and/or compartment connected thereto, amplification reagents held in an amplification compartment or a channel and/or compartment connected thereto, and reagents for preparing a nucleic acid for sequencing (such as adaptor sequences and motor proteins) held in a sequencing preparation compartment or a channel and/or compartment connected thereto. In some embodiments, the reaction module is configured for controlled delivery of a necessary reagent to a requisite compartment, e.g., a reverse transcription compartment connected to a reverse transcription reagent channel. In some embodiments, the reaction module is configured with a compartment to store a reagent, wherein the reagent compartment is connected to a compartment in which the processing of a nucleic acid occurs, e.g., a reverse transcription compartment connected to a reverse transcription reagent compartment via a channel. In some embodiments, the reaction module comprises a combination of the features for reagent holding and delivery described herein. In some embodiments, the reagents in a reagent compartment are maintained in dry form. In some embodiments, the reagents in a reagent compartment are maintained in dry form, wherein the dryness is further ensured by addition of desiccant in a part of the reagent compartment or a component in fluid communication thereto. In some embodiments, the reagent compartment comprises a sub-compartment, such a sub-compartment that is thermally isolated from the reaction compartment to ensure stability of the reagents. In some embodiments, the volume of a reagent compartment is about 5 μL to about 100 μL. In some embodiments, the volume of a reagent compartment is at least about 1 μL, such as at least about any of 2 μL, 3 μL, 4 μL, 5 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, or 100 μL. In some embodiments, the volume of a reagent compartment is less than about 100 μL, such as less than about any of 95 μL, 90 μL, 85 μL, 80 μL, 75 μL, 70 μL, 65 μL, 60 μL, 55 μL, 50 μL, 45 μL, 40 μL, 35 μL, 30 μL, 25 μL, 20 μL, 15 μL, 10 μL, 5 μL, 4 μL, 3 μL, 2 μL, or 1 μL.

In some embodiments, the devices described herein are configured to subject contents in a reaction module, or a portion thereof, to one or more reaction conditions. For example, in some embodiments, the device comprises a reaction module comprising a compartment in which the temperature of contents therein can be controlled and/or monitored. In some embodiments, the device comprises a reaction module comprising a reverse transcription compartment, an amplification compartment, and a sequencing preparation compartment, of which the temperature of contents in any one or more of the compartments can be controlled and/or monitored. In some embodiments, the device comprises a heating element, wherein the device is configured such that the heating element is in close proximity to a reaction module or component thereof, such as an amplification compartment. In some embodiments, the reaction module comprises a heating element, wherein the reaction module is configured such that the heating element is in close proximity to a component of the reaction module, such as an amplification compartment. In some embodiments, the device comprises a temperature sensor, wherein the device is configured such that the temperature sensor is in close proximity to a reaction module or component thereof, such as an amplification compartment. In some embodiments, the reaction module comprises a temperature sensor, wherein the reaction module is configured such that the temperature sensor is in close proximity to a component of the reaction module, such as an amplification compartment. In some embodiments, the heating element is a transistor-based heating element. In some embodiments, the heating element is a resistor-based heating element. In some embodiments, the temperature sensor is a junction temperature sensor. In some embodiments, the temperature sensor is a temperature-sensing transistor. In some embodiments, the device comprises a cooler, such as a Peltier cooler, wherein the device is configured such that the cooler is in proximity to a reaction module or a component thereof.

In some embodiments, the device is configured to subject the contents in a reaction module or a portion thereof, such as an amplification compartment, to a temperature of about 20° C. to about 100° C. In some embodiments, the device is configured to subject the contents in an enrichment module or a portion thereof, such as the enrichment compartment, to a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., 95° C., or 100° C. In some embodiments, the device is configured to subject the contents in a reaction module or a portion thereof, such as an amplification compartment, to cycle of temperatures of about 30° C. to about 100° C., such as about 50° C. to about 90° C.

In some embodiments, the reaction module is configured to allow for detection of components therein. For example, in some embodiments, the reaction module comprises a compartment that is configured to allow for detection of a reagent therein via, e.g., light (such as fluorescence). In some embodiments, the reaction module comprises an amplification compartment that is configured to allow for detection of a fluorescent reagent therein. In some embodiments, the reaction module comprises a detector (such as a light sensor, e.g., a fluorescence detector), wherein the reaction module is configured such that the detector is in proximity to a compartment, such as an amplification compartment. In some embodiments, the device comprises a detector (such as a light sensor, e.g., a fluorescence detector), wherein the device is configured such that the detector is in proximity to a compartment such as an amplification compartment, of a reaction module.

iv. Nucleic Acid Sequencers

The devices described herein comprise a nucleic acid sequencer. In some embodiments, the nucleic acid sequencer is a real-time nucleic acid sequencer. In some embodiments, the real-time nucleic acid sequencer detects measurable signals (light, heat, electric current, magnetic field).

In some embodiments, the nucleic acid sequencer is a nanopore sequencer. Nanopore sequencers and methods of use thereof are known in the art, e.g., see, International Application Publication Nos. WO2001042782, WO2008124706, WO2014064443, WO2018236906; U.S. Pat. No. 8,673,556; and Sutten et al., Sci Rep, 9, 2019, each of which are incorporated by reference in their entirety. In some embodiments, the nanopore sequencer comprises a nanopore array that utilizes pores (e.g., a hollow structure discrete from and extending across a barrier, such as a membrane) that permit ions, electric current, and/or fluids to cross from one side of a barrier (such as a membrane) to the other side of the barrier. For example, a nanopore array may comprise a membrane that inhibits the passage of ions or water soluble molecules can include a nanopore structure that extends across the membrane to permit the passage of, e.g., the ions or water soluble molecules from one side of the membrane to the other side of the membrane. Examples of the nanopore include, for example, biological nanopores, solid state nanopores, and biological and solid state hybrid nanopores. In some embodiments, on either side of the membrane is a pool of a medium, such as an electrically conductive medium. In some embodiments, a voltage difference (such as ranging from about −1 V to about 1V) can be imposed across the membrane between the pools of media, and conductance across the pore is determined by measuring the flow of current across the pore via the conducting medium (e.g., by ionic flow measurements). In some embodiments, the pools on either side of the membrane may be referred to as the cis pool and the trans pool. In some embodiments, one or more nanopores fluidically connect the cis pool and trans pool (which are separated by a barrier such as a membrane). Alternatively, an electrochemical gradient may be established by a difference in the ionic composition of the two pools of medium, either with different ions in each pool, or different concentrations of at least one of the ions in the solutions or media of the pools. In some embodiments, the conductance changes are measured and are indicative of monomer-dependent characteristics.

In some embodiments, when individual nucleotides of a nucleic acid interact with the pore, changes in current can be measured, thereby allowing identification of the nucleotide. For example, in some embodiments, the nucleic acid is sequenced using a nanopore sequencer, the nanopore sequencer comprising a nanopore array comprising two separate, adjacent pools of a medium and an interface (e.g., a lipid bilayer) between the two pools, the interface having a pore or channel (e.g., bacterial porin molecule) so dimensioned as to allow sequential monomer-by-monomer passage from one pool to another of only one nucleic acid polymer at a time, wherein sequencing is performed by (a) placing the nucleic acid to be sequenced in one of the two pools; and (b) taking measurements (e.g., ionic flow measurements, including measuring duration or amplitude of ionic flow blockage) as each of the nucleotide monomers of the nucleic acid passes through the pore or channel, so as to determine the sequence of the nucleotides in the nucleic acid polymer. In some embodiments, the interface can include more than one channel or pore. In some embodiments, the nucleic acid can interact with an inner surface of the channel or pore.

In some embodiments, the density of pores of a nanopore array is about 1 pores per mm² of the barrier (such as a membrane) to about 5,000 pores per mm² of the barrier. In some embodiments, the nanopore array comprises about 1 pore to about 5,000 pores, such as about 500 pores to about 525 pores, or about 2,000 pores to about 2050 pores.

In some embodiments, the nanopore array of the nanopore sequencer is fluidically connected to a reaction module, such as with a channel, e.g., a microfluidic channel. In some embodiments, the nanopore sequencer comprises a sample input port. In some embodiments, the nanopore sequencer or a portion thereof is connected to a priming channel. In some embodiments, the nanopore sequencer or a portion thereof is connected to a priming reagent compartment via a priming channel.

In some embodiments, the priming reagent compartment comprises a volume of about 10 μL to about 100 μL. In some embodiments, the volume of a priming reagent compartment is at least about 10 μL, such as at least about any of 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, or 100 μL. In some embodiments, the volume of a priming reagent compartment is less than about 100 μL, such as less than about any of 95 μL, 90 μL, 85 μL, 80 μL, 75 μL, 70 μL, 65 μL, 60 μL, 55 μL, 50 μL, 45 μL, 40 μL, 35 μL, 30 μL, 25 μL, 20 μL, 15 μL, or 10 μL. In some embodiments, additional compartments are connected to the priming compartment to provide an ability to reuse a nanopore sequencer for multiple consecutive samples. The additional compartments may contain a flow cell wash buffer and additional volume of the priming buffer. In some embodiments, the additional compartment has a volume of about 1 to about 10 volumes of the priming reagent compartment.

In some embodiments, the device comprises an on-board controller for operating the nucleic acid sequencer. In some embodiments, the device comprises an on-board user interface for operating the nucleic acid sequencing. In some embodiments, the device comprises a connection port for connecting to an off-device controller for operating the nucleic acid sequencer.

v. Fluid Movement

The devices described herein comprise a fluidic circuit comprising, e.g., an enrichment module, a reaction module, and a nucleic acid sequencer, and are capable of moving one or more fluids throughout the fluidic circuit in a controlled manner, and for moving a fluid from the fluidic circuit to a nucleic acid sequencer. Components for controlling fluid movement through and from a portion of a fluidic circuit are known in the art, e.g., see U.S. Patent Application Nos. 20180304260, 20160310948, and 20180297026, each of which are incorporated by reference in their entirety.

In some embodiments, the device comprises one or more features to control fluid movement through a portion of the fluidic circuit using, in whole or in part, a pressure and/or gravity. In some embodiments, device controls fluid flow through the fluidic circuit by controlling any one or more of venting, pumping, and valving of or in the fluidic circuit. In some embodiments, any one or more of the vent, pump, and valve is in fluidic communication with at least a portion of the fluidic circuit. In some embodiments, device controls fluid flow through the fluidic circuit via a pneumatic force.

In some embodiments, the devices described herein comprise connective means, such as a channel, to connect two or more features of a fluidic circuit. In some embodiments, connective means include, but are not limited to, fluidic channels, valves, tubes, capillaries, or other structures capable to provide uninterrupted connection of liquids. In some embodiments, connective means include physical devices capable of moving droplets of liquid that be may conductive or dielectric in reflection to a change in electric field.

In some embodiments, the device or a component thereof, such as a fluidic circuit, comprises a flow sensor.

vi. Cartridge Configurations of the Devices Described Herein

In some aspects, the devices described herein are configured such that at least one component is housed in a cartridge configured to be inserted into the device. In some embodiments, the cartridge is a disposable cartridge. The cartridges encompassed by the present disclosure are designed to be inserted in the devices described herein, and may interface with one or more other cartridges and/or other features of the device. In some embodiments, the device is configured to assess identity and placement of a cartridge therein. For example, in some embodiments, a cartridge comprises a barcode tag, RFID tag or magnetic tag, and the device comprises an associated reader capable of identifying and/or assessing the placement of a cartridge.

In some embodiments, the enrichment module, the reaction module, and the nucleic acid sequencer are housed in a single cartridge. In some embodiments, the cartridge further comprises a sample preparation module. In some embodiments, the sample preparation module is housed in another cartridge.

In some embodiments, the enrichment module and the reaction module are housed in a first cartridge, and the nucleic acid sequencer is housed in a second cartridge. In some embodiments, the first cartridge further comprises a sample preparation module. In some embodiments, the sample preparation module is housed in a third cartridge.

In some embodiments, the cartridge is configured or comprises a feature useful to interface the cartridge with another cartridge and/or another feature of the device. In some embodiments, the cartridges described herein may comprise any one or more of temperature control elements (such as heating elements), sensors (such as temperature sensors), detectors (such as fluorescence detectors), and components for controlling fluidic movement. In some embodiments, the cartridges described herein are configured to interface with any one or more of the following features housed on the device, such as temperature control elements (such as heating elements), sensors (such as temperature sensors), detectors (such as fluorescence detectors), and components for controlling fluidic movement (such as pumps).

vii. Other Components of or for Use with the Devices Described Herein

The devices and components thereof are useful for determining a target nucleic acid profile of a sample. Accordingly, the devices comprise or interface with features for operating method steps involved in determining a target nucleic acid profile of a sample. In some embodiments, the device comprises and/or interfaces with a user interface for operating the device, such as for selecting and running a method for determining a target nucleic acid profile of a sample. In some embodiments, the device comprises and/or interfaces with a control module, e.g., a computer capable of executing instructions for determining a target nucleic acid profile of a sample (such as instructions for fluid control, reaction temperature control, and sequencer control).

In some embodiments, the device comprises a pump configured to control fluid movement, such as in a fluidic circuit.

In some embodiments, the reagent buffer, such as a wash buffer, elution buffer, and reaction buffer is contained in an insertable container. In some embodiments, the insertable container is configured to be inserted into a cartridge or device described herein. In some embodiments, the insertable container is disposable.

In some embodiments, the device comprises a blood vial holder. In some embodiments, the device is configured to accept insertion of a blood vial and draw sample therefrom.

viii. Materials Useful for the Devices Described Herein

The devices and components thereof described herein are capable of performing various reactions. Accordingly, aspects of the devices are subjected to numerous reagents and reaction conditions. The devices disclosed herein thus are constructed from materials suitable for such an intended use. In some embodiments, the material is a solvent resistant material. In some embodiments, the material is tolerant of an extreme pH (such as ≤3 and/or ≥12). Fluidic devices, and materials thereof, are disclosed in, e.g., Chan et al., J Anal Methods Chem, ID175457, 2014; Patel et al., J Micromech Microeng, 18, 2008; and RM van DAM, Chapter 3 Solvent-Resistant Microfluidics, California Institute of Technology Thesis, 2006.

In some embodiments, the material is substantially free of nucleic acids. In some embodiments, the material is a polymer materials, such as a plastic material particularly suitable for molding such as thermosetting plastics (e.g., common polymers like epoxy and phenolic) or thermoplastic (e.g., nylon, polyethylene, and polystyrene).

### Exemplary Devices

In some aspects, provided herein is a device for determining a target nucleic acid profile of a sample, the device comprising: (a) an enrichment module, wherein the enrichment module comprises an enrichment compartment, wherein the enrichment compartment contains a capture medium, wherein capture probes are associated with the capture medium, wherein each capture probe comprises an oligonucleotide, wherein the enrichment module comprises one or more wash buffer compartments, wherein the one or more wash buffer compartments are connected to the enrichment compartment via one or more buffer channels, wherein the enrichment module comprises an elution buffer compartment, and wherein the elution buffer compartment is connected to the enrichment compartment via the elution buffer channel; (b) a reaction module, wherein the reaction module comprises a first reaction compartment, wherein the first reaction compartment is configured for any one or more of a reverse transcriptase step, an amplification step, an amplification product detection step, and a step for preparing nucleic acids for sequencing; and (c) a nucleic acid sequencer. In some embodiments, the oligonucleotide comprises one or more modified nucleotides, such as an LNA nucleotide. In some embodiments, the enrichment module further comprises a pH neutralization module. In some embodiments, the reagent module further comprises a reagent compartment, wherein the reagent compartment is connected to the first reaction compartment via the reagent channel. In some embodiments, the reagent compartment comprises one or more reagents. In some embodiments, the first reaction compartment comprises one or more reagents. In some embodiments, the enrichment module, the reaction module, and the nucleic acid sequencer are housed in a single cartridge. In some embodiments, the enrichment module and the reaction module are housed in a single cartridge, and the nucleic acid sequencer is housed in another cartridge.

In some aspects, provided herein is a device for determining a target nucleic acid profile of a sample, the device comprising: (a) an enrichment module, wherein the enrichment module comprises an enrichment compartment, wherein the enrichment compartment contains a capture medium, wherein capture probes are associated with the capture medium, wherein each capture probe comprises an oligonucleotide, wherein the enrichment module comprises one or more wash buffer compartments, wherein the one or more wash buffer compartments are connected to the enrichment compartment via one or more buffer channels, wherein the enrichment module comprises an elution buffer compartment, and wherein the elution buffer compartment is connected to the enrichment compartment via the elution buffer channel; (b) a reaction module, wherein the reaction module comprises a first reaction compartment, wherein the first reaction compartment is configured for any one or more of a reverse transcriptase step, an amplification step, an amplification product detection step, wherein the reaction module comprises a sequencing preparation compartment configured for a step for preparing nucleic acids for sequencing; and (c) a nucleic acid sequencer. In some embodiments, the oligonucleotide comprises one or more modified nucleotides, such as an LNA nucleotide. In some embodiments, the enrichment module further comprises a pH neutralization module. In some embodiments, the reagent module further comprises a reagent compartment, wherein the reagent compartment is connected to the first reaction compartment via the reagent channel. In some embodiments, the reagent compartment comprises one or more reagents. In some embodiments, the first reaction compartment comprises one or more reagents. In some embodiments, the enrichment module, the reaction module, and the nucleic acid sequencer are housed in a single cartridge. In some embodiments, the enrichment module and the reaction module are housed in a single cartridge, and the nucleic acid sequencer is housed in another cartridge.

In some aspects, provided herein is a device for determining a target nucleic acid profile of a sample, the device comprising: (a) an enrichment module, wherein the enrichment module comprises an enrichment compartment, wherein the enrichment compartment contains a capture medium, wherein capture probes are associated with the capture medium, wherein each capture probe comprises an oligonucleotide, wherein the enrichment module comprises one or more wash buffer compartments, wherein the one or more wash buffer compartments are connected to the enrichment compartment via one or more buffer channels, wherein the enrichment module comprises an elution buffer compartment, and wherein the elution buffer compartment is connected to the enrichment compartment via the elution buffer channel; (b) a reaction module, wherein the reaction module comprises a first reaction compartment, wherein the first reaction compartment is configured for a reverse transcriptase step, wherein the reaction module comprises an amplification compartment configured for an amplification step and an amplification product detection step, wherein the reaction module comprises a sequencing preparation compartment configured for a step for preparing nucleic acids for sequencing; and (c) a nucleic acid sequencer. In some embodiments, the oligonucleotide comprises one or more modified nucleotides, such as an LNA nucleotide. In some embodiments, the enrichment module further comprises a pH neutralization module. In some embodiments, the reagent module further comprises a reagent compartment, wherein the reagent compartment is connected to the first reaction compartment via the reagent channel. In some embodiments, the reagent compartment comprises one or more reagents. In some embodiments, the first reaction compartment comprises one or more reagents. In some embodiments, the enrichment module, the reaction module, and the nucleic acid sequencer are housed in a single cartridge. In some embodiments, the enrichment module and the reaction module are housed in a single cartridge, and the nucleic acid sequencer is housed in another cartridge.

### C. Methods of Using the Devices Disclosed Herein

In some aspects, provided herein are methods for determining a nucleic acid profile of a sample. In some embodiments, the method comprises (a) analyzing the sample using any one of the devices of described herein, thereby determining the nucleic acid profile of the sample. In some embodiments, the nucleic acid profile comprises information regarding the presence, or lack thereof, of a nucleic acid, and/or information regarding the sequence of the nucleic acid. As discussed herein, the devices described in the present disclosure may be configured for different purposes. Accordingly, it will be readily recognized that the methods described herein may be designed based on the device and/or the desired nucleic acid profile (and the required processing and analysis needed to assess for such a nucleic acid profile). Disclosure of method steps in a modular fashion is not to be construed as limiting the scope of the envisioned methods for determining a nucleic acid profile.

In some embodiments, the method, from introducing the sample to a device to completion of nucleic acid sequencing, is completed in less than about 8 hours, such as less than about any of 7 hours, 6 hours, 5 hours, 4 hours, 3 hours, 2 hours, or 1 hour. In some embodiments, the method, from introducing the sample to a device to completion of nucleic acid sequencing, is completed in about any of 8 hours, 7 hours, 6 hours, 5 hours, 4 hours, 3 hours, 2 hours, or 1 hour. In some embodiments, the method, from introducing the sample to a device to completion of nucleic acid sequencing, is completed in about 1 hour to about 8 hours. In some embodiments, the integrated devices described herein are capable of completing an analysis, from sample collection to the receipt of diagnostic results, in a time of less than about 4 hours, such as less than about any of 3 hours, 2 hours, or 1 hour.

In some embodiments, the method of using the devices disclosed herein are capable of achieving a sensitivity of at least about 10 cfu/mL of sample, such as at least about any of 9 cfu/mL of sample, 8 cfu/mL of sample, 7 cfu/mL of sample, 6 cfu/mL of sample, 5 cfu/mL of sample, 4 cfu/mL of sample, 3 cfu/mL of sample, 2 cfu/mL of sample, or 1 cfu/mL of sample. In some embodiments, the method of using the devices disclosed herein are capable of achieving a sensitivity of about 10 cfu/mL of sample, 9 cfu/mL of sample, 8 cfu/mL of sample, 7 cfu/mL of sample, 6 cfu/mL of sample, 5 cfu/mL of sample, 4 cfu/mL of sample, 3 cfu/mL of sample, 2 cfu/mL of sample, or 1 cfu/mL of sample.

In some embodiments, the method comprises obtaining a sample (such as a sample suspected of containing a target nucleic acid). In some embodiments, the sample is any biological sample from an individual. In some embodiments, the sample is a sterile sample. In some embodiments, the sample is a blood sample. In some embodiments, the sample is a whole blood sample. In some embodiments, the sample is a serum sample. In some embodiments, the sample is a plasma sample. In some embodiments, the sample is seminal fluid. In some embodiments, the sample is CNS fluid. In some embodiments, the sample is a forensic sample. In some embodiments, the sample is an environmental sample, such as a water or soil sample. In some embodiments, the sample is an agricultural sample. In some embodiments, the sample is a food sample. In some embodiments, the sample comprises a cell or cellular material, such as one or more bacteria. In some embodiments, the sample comprises a pathogen, such as a bacterium or virus. In some embodiments, the methods described herein can be performed in a manner that does not subject the user to contact with the sample or a component thereof.

In some embodiments, the sample volume is at least about 1 mL. In some embodiments, the sample volume is about 1 mL to about 15 mL, such as about 3 mL to about 10 mL. In some embodiments, the sample volume is less than about 15 mL, such as less than about 12 mL or less than about 10 mL.

In some embodiments, the sample, such as a blood sample, is collected into denaturing buffer in a container, such as a vacutainer. In some embodiments, the sample is mixed with a denaturing buffer. In some embodiments, the denaturing buffer is in the container prior to addition of the sample. In some embodiments, the denaturing buffer lyses cells, such as bacterial cells and human cells, and stabilizes nucleic acids. In some embodiments, the sample is a whole blood sample. In some embodiments, the volume of the whole blood sample is about 1 ml to about 15 ml.

In some embodiments, the sample comprises, or is suspected of comprising, a nucleic acid. In some embodiments, the nucleic acid is a deoxyribonucleic acid (DNA). In some embodiments, the nucleic acid is a ribonucleic acid (RNA), including a messenger RNA (mRNA), a ribosomal ribonucleic acid (rRNA), a transfer RNA, (tRNA), a small nuclear (snRNA), a telomerase associated RNA, or a ribozyme associated RNA. In some embodiments, the nucleic acid may be provided in a complex biological mixture of nucleic acid (RNA, DNA and/or rRNA) and non-nucleic acid. In some embodiments, the nucleic acid is one or more RNA molecules. In some embodiments, the nucleic acid is rRNAs, such as the 16S or 23S rRNA.

In some embodiments, the method comprises preparing a sample for nucleic acid enrichment. In some embodiments, preparing a sample occurs outside or within the device. In some embodiments, preparing a sample comprises subjecting the sample to a buffer useful for sample processing (such as cell lysis and nucleic acid preservation). In some embodiments, preparing a sample comprises subjecting the sample to a lysing agent, e.g., a cell/virus lysis buffer or denaturing buffer. In some embodiments, the lysing agent is a chaotropic agent. In some embodiments, preparing a sample comprises subjecting the sample to a stabilizing agent, e.g., an agent that denatures nuclease enzymes. For example, a denaturing buffer will serve to lyse both bacterial and human cells that are present as well as stabilizing nucleic acids in the sample by denaturing nuclease enzymes. Nucleic acids, such as RNA, may be highly transient substances and can be rapidly broken down outside and in cells, and upon lysis many cells release high levels of nuclease enzymes. As envisioned for the methods described herein, to increase sensitivity and enable detection of low levels of RNA, cells in a sample must be quickly lysed and the RNA preserved. This lysis process also serves to release components of bacterial cells and nucleic acids present inside human white blood cells that have engulfed bacteria. Without lysis, the typical process of degradation inside the white blood cell would proceed for some time during transport of the sample, thereby reducing the amount of detectable RNA in the specimen by the time it is tested. In some embodiments, preparing a sample comprises subjecting the sample to a condition to lyse components therein, such as one or more of sonication, agitation, or temperature.

In some embodiments, the method comprises subjecting the sample to an enrichment procedure, such as an enrichment procedure performed within the device. In some embodiments, the enrichment procedure comprises subjecting the sample to an enrichment compartment that contains a capture medium, wherein capture probes are associated with the capture medium, and wherein each capture probe comprises an oligonucleotide. In some embodiments, the sample is subjected to a capture medium, wherein capture probes are associated with the capture medium, for at least 2 cycles, such as at least any of 5 cycles, 10 cycles, 15 cycles, 20 cycles, 25 cycles, 30 cycles, or 35 cycles. In some embodiments, the enrichment procedure comprises subjecting a capture medium, wherein capture probes are associated with the capture medium, to a wash buffer. In some embodiments, the capture medium is subjected to a wash buffer prior to subjecting the capture medium to a sample. In some embodiments, the capture medium is subjected to a wash buffer after subjecting a wash buffer to a sample. In some embodiments, the capture medium is subjected to one or more wash buffers both before and after subjecting a wash buffer to a sample. Wash buffers are known in the art, and include, e.g., 3M guanidinium thiocyanate, 10 mM Tris-HCl (pH 8.5), 1% Triton X-100, or 150 mM NaCl, 100 mM Tris-HCl (pH 8.5), 0.02% Triton X-100, or 150 mM NaCl, 50 mM Tris-HCl (pH 8.5).

In some embodiments, the methods described herein comprise use of a plurality of consecutive wash buffers, such as 2, 3, or 4 or more. In some embodiments, the first wash buffer represents a buffer, similar in composition to the sample-containing buffer. In some embodiments, additional wash buffer compositions can be used to reduce residual contamination by components of the sample and to improve chemical compatibility with the elution buffer (e.g., prevent precipitation of buffer components). Volume of wash buffers may be proportional to the size of the enrichment compartment and represent, e.g., 1-200 times the volume of the enrichment compartment or capture medium.

In some embodiments, the enrichment procedure comprises subjecting the capture medium, wherein capture probes are associated with the capture medium, to an elution buffer. In some embodiments, the elution buffer has a pH of about 10 or greater, such as about 10.1 or greater, about 10.2 or greater, about 10.3 or greater, about 10.4 or greater, about 10.5 or greater, about 10.6 or greater, about 10.7 or greater, about 10.8 or greater, about 10.9 or greater, about 11 or greater, about 11.1 or greater, about 11.2 or greater, about 11.3 or greater, about 11.4 or greater, about 11.5 or greater, about 11.6 or greater, about 11.7 or greater, about 11.8 or greater, about 11.9 or greater, about 12 or greater, about 12.1 or greater, about 12.2 or greater, about 12.3 or greater, about 12.4 or greater, about 12.5 or greater, about 12.6 or greater, about 12.7 or greater, about 12.8 or greater, about 12.9 or greater, about 13 or greater, about 13.1 or greater, about 13.2 or greater, about 13.3 or greater, about 13.4 or greater, about 13.5 or greater, about 13.6 or greater, about 13.7 or greater, about 13.8 or greater, about 13.9 or greater, or about 14 or greater. In some embodiments, the elution buffer has a pH of about any of 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, or 14.

In some embodiments, the elution buffer has a pH of about 4 or less, such as about 3.9 or less, about 3.8 or less, about 3.7 or less, about 3.6 or less, about 3.5 or less, about 3.4 or less, about 3.3 or less, about 3.2 or less, about 3.1 or less, about 3 or less, about 2.9 or less, about 2.8 or less, about 2.7 or less, about 2.6 or less, about 2.5 or less, about 2.4 or less, about 2.3 or less, about 2.2 or less, about 2.1 or less, about 2 or less, about 1.9 or less, about 1.8 or less, about 1.7 or less, about 1.6 or less, about 1.5 or less, about 1.4 or less, about 1.3 or less, about 1.2 or less, about 1.1 or less, about 1 or less, about 0.9 or less, about 0.8 or less, about 0.7 or less, about 0.6 or less, about 0.5 or less, about 0.4 or less, about 0.3 or less, about 0.2 or less, or about 0.1 or less. In some embodiments, the elution buffer has a pH of about any of 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1.

In some embodiments, the volume of elution buffer subjected to the capture medium is less than about 100 μL, such as less than about any of 75 μL, 50 μL, or 25 μL.

In some embodiments, the enrichment procedure comprises subjecting a nucleic acid to an elution buffer at an extreme pH, such as an elution buffer at a pH of about <3 or >11, for less than about 10 minutes, such as less than about any of 9 minutes, 8, minutes, 7 minutes, 6 minutes, 5 minutes, 4 minutes, 3 minutes, 2 minutes, 1 minute, or 30 seconds. In some embodiments, the enrichment procedure comprises subjecting an eluate from an enrichment compartment to a pH neutralization step. In some embodiments, the pH neutralization step comprises adding a buffer (such as a having a desired pH and at a desire quantity) to neutralize the eluate. In some embodiments, the pH neutralization step comprises measuring (such as monitoring) the pH of a fluid. In some embodiments, the pH neutralization step comprises mixing an eluate from an enrichment compartment to a buffer until a desired pH is reached, wherein the pH is measured (such as monitored).

In some embodiments, the enrichment procedure comprises directed fluid from the enrichment compartment to a waste compartment (such as sample and wash buffer that has pass through the enrichment compartment).

In some embodiments, the method described herein comprises subjecting an eluate from an enrichment compartment to an amplification procedure. In some embodiments, the amplification procedure comprises subjecting a fluid comprising or suspected of comprising a nucleic acid to a reverse transcription step, which comprises subjecting the fluid to a reverse transcriptase in a condition suitable to produce cDNA. In some embodiments, the amplification procedure comprises subjecting a fluid comprising or suspected of comprising a nucleic acid to an amplification step. Reverse transcription steps and amplification steps are well known in the art, e.g., see L. Garibyan et al., J Invest Dermatol, 133, 2013; P. Kralik et al., Front Microbiol, 8, 2017; and P. Gill et al., Nucleosides, Nucleotides and Nucleic Acids, 27, 2008, each of which are incorporated by reference in their entirety. In some embodiments, the amplification step includes, but is not limited to PCR (such a qPCR), Reverse Transcription-PCR (RT-PCR), Loop-mediated amplification (LAMP), RT-LAMP, Transcription-mediated amplification (TMA), Strand displacement amplification (SDA), NASBA, and Recombination-mediated amplification (RMA).

Reverse transcription and amplification conditions and reagents are known in the art. In some embodiments, the amplification procedure comprises subjecting a fluid to an elevated temperature (including cycling at elevated temperatures), e.g., a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., 95° C., or 100° C. In some embodiments, the amplification procedure comprises cycling a fluid at elevated temperatures, e.g., cycling of temperatures of about 30° C. to about 100° C., such as about 60° C. to about 90° C. In some embodiments, the amplification procedure comprises subjecting a fluid to at least about 15 amplification cycles, such as at least about any of 20 amplification cycles, 25 amplification cycles, 30 amplification cycles, 35 amplification cycles, 40 amplification cycles, 45 amplification cycles, 50 amplification cycles, 55 amplification cycles, 60 amplification cycles, 65 amplification cycles, 70 amplification cycles, 75 amplification cycles, 80 amplification cycles, 85 amplification cycles, 90 amplification cycles, 95 amplification cycles, or 100 amplification cycles.

In some embodiments, the method described herein comprises detecting amplification products. Methods of detecting amplification products are known in the art, and include, e.g., quantitative PCR. See, e.g., S N Perison et al., Methods Mol Biol, 362, 2007. In some embodiments, the method comprises determining the presence (including the level) of a nucleic acid.

In some embodiments, the methods described herein comprise subjecting amplification products to one or more steps for preparing a nucleic acid for nucleic acid sequencing. In some embodiments, the one or more steps for preparing a nucleic acid for nucleic acid sequencing comprise adding an adaptor to the nucleic acid sequence. In some embodiments, the one or more steps for preparing a nucleic acid for nucleic acid sequencing comprise subjecting the nucleic acid to a motor protein. In some embodiments, the one or more steps for preparing a nucleic acid for nucleic acid sequencing comprise adding an adaptor to the nucleic acid sequence, and subjecting the nucleic acid to a motor protein.

In some embodiments, the method described herein comprises subjecting amplification products to nucleic acid sequencing. In some embodiments, the amplification products of an amplification reaction are subjected to nucleic acid sequencing if the presence (such as a level) of the amplification product is reached. In some embodiments, the nucleic acid sequencing provides real-time sequencing information. In some embodiments, the nucleic acid sequencing is performed using a nanopore sequencer.

As discussed herein, in some embodiments, aspects of a device may be contained in one or more cartridges. In some embodiments, the method comprises selecting one or more cartridges for use in determining a target nucleic acid profile of a sample, wherein the one or more cartridges are selected based on any of: a characteristic of the sample (e.g., source, type, volume, measured/suspected/estimated amount of a target nucleic acid), the target nucleic acid(s), the desired target nucleic acid profile, the nucleic acid sequencer, the device, environmental conditions, speed, specificity, and sensitivity.

In some embodiments, provided herein is a method to perform diagnostic test comprising: a) obtaining a sample of biological specimen from a patient, b) processing biological sample to render a subset of nucleic acids present in the said sample amplifiable in nucleic acid amplification reaction, c) performing nucleic acid amplification to amplify said nucleic acids, d) combining amplified nucleic acids with accessory reagents to real-time nucleic acid sequencing, e) conducting real-time nucleic acid sequencing, f) deriving nucleic acid sequences from results of sequencing, g) providing patient diagnosis based on the sequencing results, wherein processes of b)-e) are performed within fluidically connected compartments.

In some embodiments, provided herein is a method to perform diagnostic test comprising: a) obtaining a sample of biological specimen from a patient, b) processing biological sample to release nucleic acids from cells or complexes with proteins, c) performing nucleic acid amplification to amplify released nucleic acid, d) combining amplified nucleic acids with accessory reagents to conduct nanopore-based sequencing, e) conducting nanopore-based sequencing, f) deriving nucleic acid sequences from results of sequencing, g) providing patient diagnosis based on the sequencing result, wherein processes of b)-e) are operationally connected by sequential fluid transfer and are performed within a single consumable device.

In some embodiments, provided herein is a method to perform diagnostic test comprising: a) obtaining a sample of biological specimen from a patient, b) processing biological sample to release nucleic acids from cells or complexes with proteins, c) modifying released nucleic acids rendering them acceptable for nanopore-based sequencing, d) conducting nanopore-based sequencing, e) deriving nucleic acid sequences from results of sequencing, f) providing patient diagnosis based on the sequencing result, wherein processes of b-e are operationally connected by sequential fluid transfer and are performed within a single consumable device.

In some embodiments, the process of diagnostic testing is completed within 4 hours. In some embodiments, the single consumable device is pre-filled with reagents to perform a complete diagnostic test. In some embodiments, operational connection is provided by pressure driven fluid transfer. In some embodiments, operational connection is provided by fluid transfer enabled by change in electric field. In some embodiments, operational connection is provided by driven fluid transfer enabled by change in electric field. In some embodiments, at least two of the steps b-e are performed at two different temperatures.

In the methods of using the devices described herein, sample of biological specimen comprises biological material containing single or double stranded nucleic acids sample such as a body tissue, a biopsy, a skin sample, blood, serum, plasma, sweat, saliva, cerebrospinal fluid, mucus, uterine lavage fluid, a vaginal swab, a pap smear, a nasal swab, an oral swab, a tissue scraping, hair, a finger print, urine, stool, vitreous humor, peritoneal wash, sputum, bronchial lavage, oral lavage, pleural lavage, gastric lavage, gastric juice, bile, pancreatic duct lavage, bile duct lavage, common bile duct lavage, gall bladder fluid, synovial fluid, an infected wound, a non-infected wound, an archaeological sample, a forensic sample, a tissue sample, a food sample a bacterial sample, a protozoan sample, a fungal sample, an animal sample, a viral sample, a multi-organism sample, a fingernail scraping, semen, prostatic fluid, vaginal fluid, a vaginal swab, a fallopian tube lavage, a cell free nucleic acid, a nucleic acid within a cell, a lavage or a swab of an implanted foreign body, a nasal lavage, intestinal fluid, epithelial brushing, epithelial lavage, an autopsy sample, a necropsy sample, an organ sample, a human identification sample.

Sample of biological specimen can be collected into a variety of collection devices permitting storage and transfer of biological material without loss of single or double stranded nucleic acids.

The process of releasing nucleic acids from the sample comprises lysis of biological specimen and nucleic acid purification or enrichment.

The term “lysis” as used herein refers to the disruption, degradation and/or digestion of the biological sample. In a respective lysis step, nucleic acids can be released from cells or can be freed from other sample components such as e.g. proteins. Herein, it is referred to a respective step to disrupt, degrade and/or digest a biological sample generally as lysis step, irrespective of whether nucleic acids are released from cells or whether the lysis is performed in order to release nucleic acids e.g. from proteins or other substances comprised in the biological sample, which may also be a cell-free or cell depleted sample such as plasma. Several methods are known in the prior art that allow to achieve an efficient lysis of different sample materials. Suitable lysis methods include but are not limited to mechanical, chemical, physical or enzymatic actions on the sample. Examples of respective lysis steps include but are not limited to grinding the sample in a bead mill, sonication, surface acoustic waves (SAW), repeated cycles of freezing and thawing, heating, the addition of detergents and/or the addition of protein degrading compounds such as e.g. protein degrading enzymes, e.g. hydrolases or proteases or salts. According to one embodiment, a protein degrading compound is used during lysis. The protein-degrading compound preferably is a proteolytic enzyme. A proteolytic enzyme refers to an enzyme that catalyzes the cleavage of peptide bonds, for example in proteins, polypeptides, oligopeptides and peptides. Exemplary proteolytic enzymes include but are not limited to proteinases and proteases in particular subtilisins, subtilases, alkaline serine proteases and the like.

In some embodiments, aqueous lysis compositions are compatible for providing a lysate that is suitable for direct use in an amplification reaction such as a PCR. In other embodiments, nucleic acids are enriched or purified prior to the amplification.

In some embodiments, it is also advantageous to collect specimens in solution enacting lysis of biological material, liberating nucleic acids, while substantially inactivating proteins and preserving nucleic acids from degradation. Examples of such collection devices comprise Tempus RNA Tube (ThermoFisher) or eNAT collection solution and device (Copan, Italy).

In some embodiments, nucleic acids are preferably amplified in a nucleic acid amplification reaction. Examples of such amplification include, but are not limited to PCR, Reverse Transcription-PCR (RT-PCR), Loop-mediated amplification (LAMP), RT-LAMP, Transcription-mediated amplification (TMA), Strand displacement amplification (SDA), NASBA, Recombination-mediated amplification (RMA) and similar processes providing amplification of target sequence in double stranded or single stranded form.

Nucleic acid amplification can additionally incorporate specific nucleic acids or non-nucleic acid elements covalently linked to nucleotides or oligonucleotides that facilitate a process of real-time nucleic acid sequencing. For example, specific sequences and oligonucleotides with chemical modifications, such as spacer sequences, are commonly used to facilitate nanopore sequencing and can be incorporated during PCR or RT-PCR amplification step.

Real-time sequencing methods are methods capable of detecting measurable signal (light, heat, electric current, magnetic field) and interpreting it into nucleic acid sequence within the duration of a sequencing run. An example of real-time sequencing method is a nanopore-based sequencing method that measures an electric current and translates it to nucleic acid sequence.

### D. Kits, Components, and Compositions (Such as Consumables) of the Devices, or for Use with the Devices

The present disclosure also provides kits, components, and compositions (such as consumables) of the devices, or for use with the devices described herein. For example, provided herein is a cartridge comprising an enrichment module and a reaction module. In some embodiments, provided herein is a cartridge comprising an enrichment module, a reaction module, and a nucleic acid sequencer. In some embodiments, provided herein is a cartridge comprising a sample preparation module, an enrichment module, and a reaction module. In some embodiments, provided herein is a cartridge comprising a sample preparation module, an enrichment module, a reaction module, and a nucleic acid sequencer. In some embodiments, provided herein is a cartridge comprising a sample preparation module. In some embodiments, provided herein is a cartridge comprising a nucleic acid sequencer.

In some embodiments, provided herein is a reagent compartment, such as a wash buffer compartment, elution buffer compartment, or a reagent compartment.

### EXEMPLARY EMBODIMENTS

Embodiment 1. A device for determining a target nucleic acid profile of a sample, the device comprising: (a) an enrichment module, wherein the enrichment module comprises an enrichment compartment; (b) a reaction module, wherein the reaction module comprises a first reaction compartment; and (c) a nucleic acid sequencer.

Embodiment 2. The device of embodiment 1, wherein at least two of any of (i) the enrichment module, (ii) the reaction module, and (iii) the nucleic acid sequencer are connected via a fluidic circuit.

Embodiment 3. The device of embodiment 2, wherein the enrichment module is connected to the reaction module via a first channel.

Embodiment 4. The device of any one of embodiments 1-3, wherein the reaction module is connected to the nucleic acid sequencer via a second channel.

Embodiment 5. The device of any one of embodiments 1-4, wherein the enrichment compartment has a volume of about 10 mm³ to about 100 mm³.

Embodiment 6. The device of any one of embodiments 1-5, wherein the enrichment compartment contains a capture medium, wherein capture probes are associated with the capture medium, and wherein each capture probe comprises an oligonucleotide.

Embodiment 7. The device of embodiment 6, wherein the oligonucleotide comprises one or more modified nucleotides.

Embodiment 8. The device of embodiment 7, wherein each of the one or more modified nucleotides is selected from the group consisting of a locked nucleic acid (LNA), peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, a nucleotide with a modified sugar, base group, or backbone, or any combination thereof.

Embodiment 9. The device of any one of embodiments 6-8, wherein the capture medium is a solid support or a plurality of particles.

Embodiment 10. The device of any one of embodiments 6-9, wherein the capture probe is conjugated to a surface of the capture medium.

Embodiment 11. The device of any one of embodiments 1-9, wherein the enrichment module is configured to cycle a fluid through the enrichment compartment.

Embodiment 12. The device of any one of embodiments 1-11, wherein the enrichment module further comprises a first buffer channel, and wherein the first buffer channel is connected to the enrichment compartment.

Embodiment 13. The device of embodiment 12, wherein the enrichment module further comprises a first buffer compartment, and wherein the first buffer compartment is connected to the enrichment compartment via the first buffer channel.

Embodiment 14. The device of any one of embodiments 1-13, wherein the enrichment module further comprises an elution buffer channel, and wherein the elution buffer channel is connected to the enrichment compartment.

Embodiment 15. The device of any one of embodiments 14, wherein the enrichment module further comprises an elution buffer compartment, and wherein the elution buffer compartment is connected to the enrichment compartment via the elution buffer channel.

Embodiment 16. The device of any one of embodiments 1-14, wherein the enrichment module further comprises a pH neutralization module.

Embodiment 17. The device of embodiment 16, wherein the pH neutralization module comprises a pH sensor.

Embodiment 18. The device of any one of embodiments 1-17, wherein the enrichment module is connected to a first waste output channel.

Embodiment 19. The device of any one of embodiments 1-18, wherein the first reaction compartment is configured for any one or more of a reverse transcriptase step, an amplification step, an amplification product detection step, and a step for preparing nucleic acids for sequencing.

Embodiment 20. The device of any one of embodiments 1-19, wherein the reaction module further comprises a second reaction compartment, and wherein the first reaction compartment and the second reaction compartment are configured for any one or more of a reverse transcriptase step, an amplification step, an amplification product detection step, and a step for preparing nucleic acids for sequencing.

Embodiment 21. The device of embodiment 20, wherein the reaction module further comprises a third reaction compartment, and wherein the first reaction compartment, the second reaction compartment, and the third reaction compartment are configured for any one or more of a reverse transcriptase step, an amplification step, an amplification product detection step, and a step for preparing nucleic acids for sequencing.

Embodiment 22. The device of any one of embodiments 1-19, wherein the first reaction compartment is a reverse transcription compartment, wherein the reaction module further comprises an amplification compartment and a sequencing preparation compartment.

Embodiment 23. The device of embodiment 22, wherein the reverse transcription compartment is connected to the amplification compartment via a channel, and wherein the amplification compartment is connected to the sequencing preparation compartment via another channel.

Embodiment 24. The device of any one of embodiments 1-23, wherein the first reaction compartment is connected to a reagent channel.

Embodiment 25. The device of embodiment 24, wherein the reagent module further comprises a reagent compartment, wherein the reagent compartment is connected to the first reaction compartment via the reagent channel.

Embodiment 26. The device of any one of embodiments 1-25, wherein the first reaction compartment comprises one or more reagents useful for one or more of nucleic acid amplification, nucleic acid detection, and preparing nucleic acids for sequencing.

Embodiment 27. The device of any one of embodiments 1-26, further comprising a heating element, wherein the heating element is position in proximity or on the reaction module or a portion thereof.

Embodiment 28. The device of any one of embodiments 1-27, further comprising a temperature sensor, wherein the temperature sensor is position in proximity or on the reaction module or a portion thereof.

Embodiment 29. The device of any one of embodiments 1-28, further comprising a fluorescence detector.

Embodiment 30. The device of any one of embodiments 1-29, wherein the nucleic acid sequencer is a nanopore sequencer.

Embodiment 31. The device of embodiment 30, wherein the nanopore sequencer comprises a nanopore array that is fluidically connected to the reaction module via the second channel.

Embodiment 32. The device of embodiment 30 or 31, wherein the nanopore sequencer comprises a sample input port.

Embodiment 33. The device of embodiment 32, wherein the reagent module interfaces with the sample input port of the sample input port of the nanopore sequencer.

Embodiment 34. The device of any one of embodiments 31-33, wherein the device is configured to move a fluid from the reaction module to the nucleic acid sequencer.

Embodiment 35. The device of any one of embodiments 1-34, further comprising a sample preparation module.

Embodiment 36. The device of any one of embodiments 1-35, wherein the enrichment module is connected to a sample input channel.

Embodiment 37. The device of embodiment 36, wherein the enrichment module is connected to the sample preparation module via the sample input channel.

Embodiment 38. The device of any one of embodiments 1-37, wherein the enrichment module, the reaction module, and the nucleic acid sequencer are housed in a single cartridge.

Embodiment 39. The device of any one of embodiments 1-38, wherein the enrichment module and the reaction module are housed in a single cartridge, and the nucleic acid sequencer is housed in another cartridge.

Embodiment 40. A method of determining a nucleic acid profile of a sample, the method comprising analyzing the sample using any one of the devices of embodiments 1-39, thereby determining the nucleic acid profile of the sample.

Embodiment 41. The method of embodiment 40, wherein the nucleic acid profile comprises information regarding the presence, or lack thereof, of a nucleic acid, and/or information regarding the sequence of the nucleic acid.

The disclosure provided herein is further illustrated by the following examples, which are not to be construed as limiting the disclosure in scope or spirit to the specific procedures described in them.

### EXAMPLES

### Example 1

This example demonstrates a device and method for determining a target nucleic acid profile of a sample. Specifically, provided herein is a method for detecting an identifying a bacterial infection of the bloodstream.

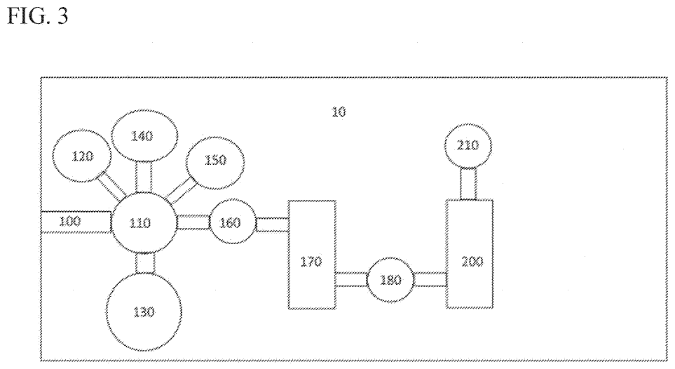

A patient sample, such as a blood sample, is collected into a Tempus RNA collection tube and subjected to cell lysis (10 minutes at 95° C.) releasing nucleic acids from bacterial cells, if present, and human cells in the presence of guanidinium salts. The prepared sample is introduced into the assay cartridge 10 (shown in FIG. 3) through a sample input port 100 and moved into the enrichment compartment 110 of the enrichment module. The enrichment compartment contains oligonucleotide capture probes covalently attached to microspheres (approximately 50-90 μm). The probes are directed against 16S bacterial ribosomal RNA sequences. Optionally, probes may be directed to polyA tailed host mRNA.

The sample is directed to flow through the enrichment compartment, which is maintained at 50° C., at a flow rate of 0.2 mL/minute and the flow through is collected in a waste compartment 130. The enrichment compartment 110 is washed with Wash 1 buffer (containing Guanidinium salts) at 50° C. (Wash 1 buffer may be held in a buffer compartment, e.g., packaged in a blister pack 120). The flow through is collected in the waste compartment 130. The enrichment compartment 110 is washed with Wash 2 buffer at 25° C. (Wash 2 buffer may be held in a buffer compartment, e.g., packaged in a blister pack 140). The flow through is collected in the waste compartment 130. The captured nucleic acids in the enrichment compartment 110 are eluted (at 20° C.) with Elution buffer (Elution buffer may be held in an elution buffer compartment, e.g., a blister pack 150. The eluate from the enrichment compartment 110 is directed to a PCR reagent compartment 160, which contains, e.g., lyophilized PCR reagents. The flow path connecting the enrichment compartment 110 and the PCR reagent compartment 160 may optionally comprise a valve.

The liquid containing dissolved PCR reagents is then directed to an amplification compartment 170, which is capable of perform thermal cycling within the temperature range of 60-90° C. The amplification compartment 170 interacts with the thermal cycler unit oriented in perpendicular plane to the main cartridge body (not shown in the picture). The thermal cycler subjects the amplification compartment 170 to 40 cycles of PCR amplification. Optionally, the amplification is monitored by a multicolor fluorescent detector to detect presence of amplified DNA products (not shown).

Liquid containing amplified DNA copies of the enriched RNA is moved to a sequencing preparation compartment 180 containing pre-dispensed accessory reagents (rapid sequencing adaptors) provided in Oxford Nanopore PCR Sequencing reagent kit SQK-PSK004 (in liquid or lyophilized form). The nanopore flow cell 200 (Minion Flow cell, Oxford Nanopore Technologies) is primed with priming buffer stored in, e.g., the blister pack 210. The prepared DNA library from the sequencing preparation compartment 180 is moved to a nanopore flow cell 200 and a sequencing protocol (using Minit sequencing controller) is initiated.

Sequencing with simultaneous base-calling interpreting current signal into DNA sequence is performed for 1-30 minutes. Obtained DNA sequences are analyzed to establish identity and frequency of detected 16S RNA reads and to interpret results for diagnostic use. Diagnostic results are provided, e.g., as positive for bacterial infection or infections in blood or negative for bacterial infection (optionally reporting detection and providing identity of contaminating microorganisms).

What is claimed is:

1. A device for determining a target nucleic acid profile of a sample, the device comprising:
(a) an enrichment module,
wherein the enrichment module comprises an enrichment compartment;
(b) a reaction module,
wherein the reaction module comprises a first reaction compartment; and
(c) a nucleic acid sequencer.

2. The device of claim 1, wherein at least two of any of (i) the enrichment module, (ii) the reaction module, and (iii) the nucleic acid sequencer are connected via a fluidic circuit.

3. The device of claim 2, wherein the enrichment module is connected to the reaction module via a first channel.

4. The device of any one of claims 1-3, wherein the reaction module is connected to the nucleic acid sequencer via a second channel.

5. The device of any one of claims 1-4, wherein the enrichment compartment has a volume of about 10 mm³ to about 100 mm³.

6. The device of any one of claims 1-5, wherein the enrichment compartment contains a capture medium, wherein capture probes are associated with the capture medium, and wherein each capture probe comprises an oligonucleotide.

7. The device of claim 6, wherein the oligonucleotide comprises one or more modified nucleotides.

8. The device of claim 7, wherein each of the one or more modified nucleotides is selected from the group consisting of a locked nucleic acid (LNA), peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, a nucleotide with a modified sugar, base group, or backbone, or any combination thereof.

9. The device of any one of claims 6-8, wherein the capture medium is a solid support or a plurality of particles.

10. The device of any one of claims 6-9, wherein the capture probe is conjugated to a surface of the capture medium.

11. The device of any one of claims 1-9, wherein the enrichment module is configured to cycle a fluid through the enrichment compartment.

12. The device of any one of claims 1-11, wherein the enrichment module further comprises a first buffer channel, and wherein the first buffer channel is connected to the enrichment compartment.

13. The device of claim 12, wherein the enrichment module further comprises a first buffer compartment, and wherein the first buffer compartment is connected to the enrichment compartment via the first buffer channel.

14. The device of any one of claims 1-13, wherein the enrichment module further comprises an elution buffer channel, and wherein the elution buffer channel is connected to the enrichment compartment.

15. The device of any one of claim 14, wherein the enrichment module further comprises an elution buffer compartment, and wherein the elution buffer compartment is connected to the enrichment compartment via the elution buffer channel.

16. The device of any one of claims 1-14, wherein the enrichment module further comprises a pH neutralization module.

17. The device of claim 16, wherein the pH neutralization module comprises a pH sensor.

18. The device of any one of claims 1-17, wherein the enrichment module is connected to a first waste output channel.

19. The device of any one of claims 1-18, wherein the first reaction compartment is configured for any one or more of a reverse transcriptase step, an amplification step, an amplification product detection step, and a step for preparing nucleic acids for sequencing.

20. The device of any one of claims 1-19, wherein the reaction module further comprises a second reaction compartment, and wherein the first reaction compartment and the second reaction compartment are configured for any one or more of a reverse transcriptase step, an amplification step, an amplification product detection step, and a step for preparing nucleic acids for sequencing.

21. The device of claim 20, wherein the reaction module further comprises a third reaction compartment, and wherein the first reaction compartment, the second reaction compartment, and the third reaction compartment are configured for any one or more of a reverse transcriptase step, an amplification step, an amplification product detection step, and a step for preparing nucleic acids for sequencing.

22. The device of any one of claims 1-19, wherein the first reaction compartment is a reverse transcription compartment, wherein the reaction module further comprises an amplification compartment and a sequencing preparation compartment.

23. The device of claim 22, wherein the reverse transcription compartment is connected to the amplification compartment via a channel, and wherein the amplification compartment is connected to the sequencing preparation compartment via another channel.

24. The device of any one of claims 1-23, wherein the first reaction compartment is connected to a reagent channel.

25. The device of claim 24, wherein the reagent module further comprises a reagent compartment, wherein the reagent compartment is connected to the first reaction compartment via the reagent channel.

26. The device of any one of claims 1-25, wherein the first reaction compartment comprises one or more reagents useful for one or more of nucleic acid amplification, nucleic acid detection, and preparing nucleic acids for sequencing.

27. The device of any one of claims 1-26, further comprising a heating element, wherein the heating element is position in proximity or on the reaction module or a portion thereof.

28. The device of any one of claims 1-27, further comprising a temperature sensor, wherein the temperature sensor is position in proximity or on the reaction module or a portion thereof.

29. The device of any one of claims 1-28, further comprising a fluorescence detector.

30. The device of any one of claims 1-29, wherein the nucleic acid sequencer is a nanopore sequencer.

31. The device of claim 30, wherein the nanopore sequencer comprises a nanopore array that is fluidically connected to the reaction module via the second channel.

32. The device of claim 30 or 31, wherein the nanopore sequencer comprises a sample input port.

33. The device of claim 32, wherein the reagent module interfaces with the sample input port of the sample input port of the nanopore sequencer.

34. The device of any one of claims 31-33, wherein the device is configured to move a fluid from the reaction module to the nucleic acid sequencer.

35. The device of any one of claims 1-34, further comprising a sample preparation module.

36. The device of any one of claims 1-35, wherein the enrichment module is connected to a sample input channel.

37. The device of claim 36, wherein the enrichment module is connected to the sample preparation module via the sample input channel.

38. The device of any one of claims 1-37, wherein the enrichment module, the reaction module, and the nucleic acid sequencer are housed in a single cartridge.

39. The device of any one of claims 1-38, wherein the enrichment module and the reaction module are housed in a single cartridge, and the nucleic acid sequencer is housed in another cartridge.

40. A method of determining a nucleic acid profile of a sample, the method comprising analyzing the sample using any one of the devices of claims 1-39,
thereby determining the nucleic acid profile of the sample.

41. The method of claim 40, wherein the nucleic acid profile comprises information regarding the presence, or lack thereof, of a nucleic acid, and/or information regarding the sequence of the nucleic acid.
